SEQUENCE LISTING (38 sequences)

SEQ ID NO: 1 - protein, 4 aa, Artificial Sequence
  Synthetic
RGDS

SEQ ID NO: 2 - protein, 5 aa, Artificial Sequence
  Synthetic
CRGDS

SEQ ID NO: 3 - protein, 6 aa, Artificial Sequence
  Synthetic
CRGDSP

SEQ ID NO: 4 - protein, 5 aa, Artificial Sequence
  Synthetic
PHSRN

SEQ ID NO: 5 - protein, 9 aa, Artificial Sequence
  Synthetic
GWGGRGDSP

SEQ ID NO: 6 - protein, 13 aa, Artificial Sequence
  Synthetic
SIDQVEPYSSTAQ

SEQ ID NO: 7 - protein, 11 aa, Artificial Sequence
  Synthetic
GRNIAEIIKDI

SEQ ID NO: 8 - protein, 9 aa, Artificial Sequence
  Synthetic
DITYVRLKF

SEQ ID NO: 9 - protein, 9 aa, Artificial Sequence
  Synthetic
DITVTLNRL

SEQ ID NO: 10 - protein, 8 aa, Artificial Sequence
  Synthetic
GRYVVLPR

SEQ ID NO: 11 - protein, 13 aa, Artificial Sequence
  Synthetic
GNRWHSIYITRFG

SEQ ID NO: 12 - protein, 12 aa, Artificial Sequence
  Synthetic
GASIKVAVSADR

SEQ ID NO: 13 - protein, 9 aa, Artificial Sequence
  Synthetic
GTTVKYIFR

SEQ ID NO: 14 - protein, 10 aa, Artificial Sequence
  Synthetic
GSIKIRGTYS

SEQ ID NO: 15 - protein, 7 aa, Artificial Sequence
  Synthetic
GSINNNR

SEQ ID NO: 16 - protein, 9 aa, Artificial Sequence
  Synthetic
SDPGYIGSR

SEQ ID NO: 17 - protein, 5 aa, Artificial Sequence
  Synthetic
YIGSR

SEQ ID NO: 18 - protein, 14 aa, Artificial Sequence
  Synthetic
GTPGPQGIAGQGVV

SEQ ID NO: 19 - protein, 14 aa, Artificial Sequence
  Synthetic
GTPGPQGIAGQRVV

SEQ ID NO: 20 - protein, 7 aa, Artificial Sequence
  Synthetic
MNYYSNS

SEQ ID NO: 21 - protein, 12 aa, Artificial Sequence
  Synthetic
KKQRFRHRNRKG

SEQ ID NO: 22 - protein, 22 aa, Artificial Sequence
  Synthetic
CRGDGGGGGGGGGGGGGPHSRN

SEQ ID NO: 23 - protein, 19 aa, Artificial Sequence
  Synthetic
CPHSRNSGSGSGSGSGRGD

SEQ ID NO: 24 - protein, 10 aa, Artificial Sequence
  Synthetic
  MOD_RES (1)..(1): ACETYLATION
GCYGRGDSPG

SEQ ID NO: 25 - protein, 5 aa, Artificial Sequence
  Synthetic
CRDGS

SEQ ID NO: 26 - protein, 5 aa, Artificial Sequence
  Synthetic
  MISC_FEATURE (4)..(4): D-amino acid
RGDFC

SEQ ID NO: 27 - protein, 12 aa, Artificial Sequence
  Synthetic
RKRLQVQLSIRT

SEQ ID NO: 28 - protein, 5 aa, Artificial Sequence
  Synthetic
IKVAV

SEQ ID NO: 29 - protein, 5 aa, Artificial Sequence
  Synthetic
YIGSR

SEQ ID NO: 30 - protein, 8 aa, Artificial Sequence
  Synthetic
KRTGQYKL

SEQ ID NO: 31 - protein, 7 aa, Artificial Sequence
  Synthetic
TYRSRKY

SEQ ID NO: 32 - protein, 17 aa, Artificial Sequence
  Synthetic
KRTGQYKLGSKTGPGQK

SEQ ID NO: 33 - protein, 14 aa, Artificial Sequence
  Synthetic
QAKHKQRKRLKSSC

SEQ ID NO: 34 - protein, 17 aa, Artificial Sequence
  Synthetic
SPKHHSQRARKKKNKNC

SEQ ID NO: 35 - protein, 6 aa, Artificial Sequence
  Synthetic
  MISC_FEATURE (1)..(1): X = hydropathic residue
  MISC_FEATURE (2)..(3): B = basic residue
  MISC_FEATURE (4)..(4): X = hydropathic residue
  MISC_FEATURE (5)..(5): B = basic residue
  MISC_FEATURE (6)..(6): X = hydropathic residue
XBBXBX

SEQ ID NO: 36 - protein, 8 aa, Artificial Sequence
  Synthetic
  MISC_FEATURE (1)..(1): X = hydropathic residue
  MISC_FEATURE (2)..(4): B = basic residue
  MISC_FEATURE (5)..(6): X = hydropathic residue
  MISC_FEATURE (7)..(7): B = basic residue
  MISC_FEATURE (8)..(8): X = hydropathic residue
XBBBXXBX

SEQ ID NO: 37 - protein, 5 aa, Artificial Sequence
  Synthetic
RGDSP

SEQ ID NO: 38 - protein, 6 aa, Artificial Sequence
  Synthetic
GSGSGS

(19) United States
(12) Patent Application Publication
(10) Pub. No.: US 2022/0295841 A1
(43) Pub. Date: Sep. 22, 2022

(54) PLANT-DERIVED SCAFFOLDS FOR GENERATION OF SYNTHETIC ANIMAL TISSUE

(71) Applicant: Wisconsin Alumni Research Foundation, Madison, WI (US)

(72) Inventors: Brian W. MOORE, Worcester, MA (US); Fatin ALKHALEDI, Worcester, MA (US); Alex S. REBELLO, Worcester, MA (US); Daniel SOCHACKI, Worcester, MA (US); William L. MURPHY, Madison, WI (US); Jordan JONES, Worcester, MA (US); Glenn GAUDETTE, Wocester, MA (US); GIANLUCA FONTANA, Madison, WI (US); Masatoshi SUZUKI, Madison, WI (US); Katherine Pearl BARTEAU, Madison, WI (US); Sin-Ruow TEY, Madison, WI (US)

(21) Appl. No.: 17/603,695

(22) PCT Filed: Apr. 17, 2020
(86) PCT No.: PCT/US2020/028782

§ 371 Date: Oct. 14, 2021

Related U.S. Application Data
(60) Provisional application No. 62/836,043, filed on Apr. 18, 2019.

(51) Int. Cl. A23L 13/00; C12N 5/00

(52) U.S. Cl. CPC A23L 13/00; C12N 5/0068; C12N 2533/90; A23V 2002/00

(57) ABSTRACT

Use of decellularized plant tissues as scaffolds for producing edible animal tissue are disclosed herein. Particularly, decellularized plant tissues are used as scaffolds for animal cells to allow growth and differentiation it animal tissue. The edible animal tissue can be dried such as in a beef jerky form for human consumption.

### CROSS-REFERENCE TO RELATED APPLICATION

This application is a national phase application of International Patent Application No. PCT/US2020/028782 (published as WO 2020/214964), filed Apr. 17, 2020, which claims priority to U.S. Provisional Application Ser. No. 62/836,043 filed Apr. 18, 2019, both of which are hereby incorporated by reference in their entireties.

### STATEMENT OF GOVERNMENT SUPPORT

This invention was made with government support under NS109427 and TR002383 awarded by the National Institutes of Health and under DMR1306482 awarded by the National Science Foundation. The government has certain rights in the invention.

### INCORPORATION OF SEQUENCE LISTING

A paper copy of the Sequence Listing and a computer readable form of the Sequence Listing containing the file named “P200267US02_ST25”, which is 8,661 bytes in size (as measured in MICROSOFT WINDOWS® EXPLORER), are provided herein and are herein incorporated by reference. This Sequence Listing consists of SEQ ID NO:1-38.

### BACKGROUND OF THE DISCLOSURE

The present disclosure relates generally to edible animal tissue, and particularly, dried meat products, such as beef jerky, prepared using decellularized plant tissues as scaffolding materials. Particularly, it was found that isolated bovine muscle cells successfully adhered to the decellularized plant leaf scaffolds, thereby exhibiting alignment, proliferation, confluence, viability, and differentiation into myocytes without the use of adherent protein coatings.

Approximately 97% of all U.S. adults consume meat on a regular basis, with the average American estimated to have consumed a record 222 pounds of red meat and poultry in the year 2018 alone. Agriculture uses 51% of all land in the United States, 80% of which is used to raise animal livestock. Because it is projected that both global population and meat production will continue to rise, there is a real risk that there will be insufficient land to keep up with the growing demand for meat.

Additionally, agriculture contributes to 24% of global greenhouse gas emissions. Experts suggest that increasing greenhouse gas levels within the Earth's atmosphere are leading to global warming. Some of the consequences of this climate change include rising ocean levels, stronger and potentially catastrophic weather events, and global drought. In addition, agriculture is responsible for consuming 70% of all freshwater globally. It is estimated that, by 2050, over half of the global population will be living in moderately water scarce areas.

It is clear that the current state of meat production and agriculture is causing large-scale environmental harm. There is a need for an alternative meat source that satisfies the growing demands of consumers, while significantly reducing land usage, greenhouse gas emissions, and water consumption. Two viable alternatives to conventional meat products include plant-based protein and cellular agriculture.

Because only 3% of U.S. citizens follow a vegan or vegetarian lifestyle, the market and environmental impact for plant-based products is relatively small. Cellular agriculture, also commonly referred to as cultured meat, is an emerging industry which utilizes tissue engineering technology to grow authentic meat products. Cellular agriculture presents a unique opportunity because it caters to the larger audience of meat eaters, and can have a significant environmental impact. One of the biggest challenges in the field of cellular agriculture, however, is the development of perfusable scaffolds that can produce structured meat.

Based on the foregoing, it would be advantageous to develop an environmentally conscious, lean, structured meat product. As described more fully herein, it was found herein that isolated bovine muscle cells successfully adhered to decellularized plant leaf scaffolds. The adhered cells exhibited alignment, proliferation, confluence, viability, and differentiation into myocytes without the use of adherent protein coatings. These results demonstrate that decellularized plant leaf technology is promising in the future production of dried meat products.

### BRIEF DESCRIPTION OF THE DISCLOSURE

The present disclosure is generally related to processes of using decellularized plant tissues scaffolding materials for growing meat. Dried meat products, such as beef jerky, are a particularly suitable use as they are a $2.8 billion industry in the United States and are currently an untapped market within cellular agriculture. Accordingly, in one aspect, the present disclosure is directed to a method of forming edible animal tissue, comprising: decellularizing plant material to form a scaffold; seeding the scaffold with animal cells; and culturing the animal cells to form a grown animal tissue from the seeded scaffold.

Additionally, the present disclosure relates to systems and methods of using the systems for bulk decellularizing the plant tissues to simplify and scale this process for growing meat on a commercial level. In particular aspects, the present disclosure is directed to a system for decellularizing a plant material, the system comprising: a device for mechanical stirring or shaking, the device capable of holding a container having an open upper end; and a tiered grate inside the container, located at the lower end, the tiered grate comprising at least two tiers, the first tier having a diameter that is smaller than the second tier.

In another aspect, the present disclosure is directed to a method of decellularizing a plant material using the system described above. The method comprises: placing the plant material into the container; contacting the plant material with one or more detergent selected from the group consisting of sodium hypochlorite (bleach), sodium dodecyl sulfate, sodium hydroxide, ethylenediaminetetraacetic acid (EDTA), Triton X-100, and the like, and combinations thereof; mechanically stirring or shaking the plant material in the container; and allowing plant material to soak in the one or more detergent for a period of from about 30 minutes to about 72 hours.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 depicts a schematic describing cannulation and decellularization of plant leaves.

FIG. 2 depicts one exemplary design of a protective grate for bulk decellularization.

FIG. 3A depicts one exemplary design of a protective tiered grate for bulk decellularization.

FIG. 3B depicts one exemplary design of a system for decellularizing plant material using the protective tiered grate of FIG. 3A.

FIG. 4A depicts the dependence of plant scaffold yield strength on decellularization treatment duration.

FIG. 4B depicts the dependence of plant scaffold toughness on decellularization treatment duration.

FIGS. 5A-5F depict mineralization of plant tissue. Clockwise, from top left are depicted: mineralized parsley stem (FIG. 5A); SEM micrograph of mineralized parsley stem (FIG. 5B); SEM micrograph of non-mineralized parsley stem (FIG. 5C); SEM micrograph of surface of mineralized bamboo stem (FIG. 5D); SEM micrograph of surface of non-mineralized bamboo stem (FIG. 5E); Faxitron image of mineral coated and non-mineral coated bamboo stem (FIG. 5F).

FIG. 6 depicts one exemplary design of seeding plant material with a carboy and handing of the plant material.

FIG. 7 depicts one exemplary design of a cell factory system for use in seeding plant material in a cell media bath.

FIG. 8 depicts one exemplary design of a system for use in seeding plant material in a cell media both agitated by a magnetic stir bar.

FIG. 9 depicts various types of leaf venation.

FIG. 10 depicts the process of carboy perfusion.

FIG. 11 depicts one exemplary design using a centrifuge for seeding cells onto the surface of a plant material.

FIG. 12 depicts the base of an incubation box design for seeding cells onto the surface of a plant material.

FIG. 13 depicts a diagram with exemplary dimensions for the incubation boxy design of FIG. 12.

FIG. 14 depicts bulk decellularization of iceberg lettuce.

FIGS. 15A & 15B depict bulk decellularization of spinach leaves.

FIG. 15C depicts bulk decellularization of leek leaves.

FIGS. 16A & 16B depict confluency of cells observed on top of spinach leaves (Actin—Green, Nuclei—Blue).

FIGS. 17A & 17B depict evidence of multinucleated myocytes on a spinach leaf (Actin—Green, Nuclei—Blue).

FIGS. 18A & 18B depict confluent monolayer of cells observed on top of iceberg lettuce leaves (Actin—Green, Nuclei—Blue).

FIGS. 19A & 19B depict cells on control plates (Actin—Green, Nuclei—Blue).

FIG. 20 depicts P9 bovine skeletal muscle cells used in seeding.

FIGS. 21A-21C depict multinucleated myocytes present in well plate (Myosin—Green, Nuclei—Blue).

FIGS. 22A & 22B depict two different areas in A2 of the 6-well plate (FIG. 22A at 10× and FIG. 22B at 20×).

FIG. 23 depicts negative staining of well D6.

FIG. 24 depicts a control well following protocol of row C.

FIGS. 25A & 25B depict control wells following protocol of row D.

FIG. 26 depicts myoblast development on a portion of an apple tree leaf as indicated by MF-20 (Green).

FIG. 27 depicts multiple markers of differentiation observed on the peach tree leaf section using MF-20 (green).

FIG. 28 depicts a cluster of MF-20 marking area of differentiation of a banana leaf.

FIG. 29 depicts (MF-20 (Green)) showing clusters of cells that are differentiated on a plum leaf.

FIG. 30 is a diagram of isolation and seeding of primary bovine satellite cells on a decellularized spinach scaffold as analyzed in Example 8.

FIGS. 31A-31C show primary bovine satellite cells viable after being cultured on decellularized spinach scaffold for 14 days as analyzed in Example 1. FIG. 31A shows Live (green)/Dead (red) staining and Hoechst (blue) staining of nuclei of primary satellite cells cultured on gelatin coated glass (control) for 14 days. FIG. 31B shows Live (green)/Dead (red) staining and Hoechst staining of nuclei (blue) of primary satellite cells cultured on decellularized spinach scaffold for 14 days. FIG. 31C is a comparison of viability percentage of primary satellite cells cultured on gelatin coated glass (control) vs. decellularized spinach scaffold.

FIGS. 32A-32C show primary bovine satellite cells differentiated on a decellularized spinach scaffold after 14 days. FIG. 32A shows myosin heavy chain (MHC) staining (green) and Hoechst staining of nuclei (blue) of primary satellite cells cultured on gelatin coated glass (control) for 14 days. FIG. 32B shows MHC staining (green) and Hoechst staining of nuclei (blue) of primary satellite cells cultured on decellularized spinach scaffold for 14 days. FIG. 32C shows a comparison of differentiation percentage of primary satellite cells cultured on gelatin coated glass (control) vs. decellularized spinach scaffold.

FIGS. 33A-33D show some cell-loaded scaffolds demonstrated alignment among seeded cells. FIG. 31A shows Phalloidin staining of F-actin microfilaments (green) and Hoechst staining of nuclei of primary bovine satellite cells cultured on gelatin coated glass (control) and decellularized spinach scaffold for 14 days. FIG. 31B shows directional analysis color survey indicating the direction of each microfilament of primary bovine satellite cells cultured on gelatin coated glass (control) and decellularized spinach scaffold for 14 days. FIG. 31C shows a comparison of alignment of primary satellite cells cultured on gelatin coated glass (control) vs. decellularized spinach scaffold. FIG. 31D depicts direction distribution of microfilaments of primary bovine satellite cells cultured on gelatin coated glass (control) and decellularized spinach scaffold for 14 days.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

In general, the present disclosure is directed to methods of preparing edible animal tissue, and particularly, cultured meat products using decellularized plant tissues as scaffolding material. Particularly, the present disclosure is directed to methods of forming edible animal tissue including: decellularizing vascular edible plant material to form a scaffold; seeding the scaffold with animal cells; and harvesting an animal tissue from the seeded scaffold.

Cellular agriculture is an alternative method for growing clean or cultured meat, and is defined as the process of creating edible animal muscle-skeletal tissue in vitro using tissue engineering techniques. With increasing public awareness of the ethical, environmental, and sustainability concerns surrounding the animal agriculture industry, cultured meat is an ecological alternative to satisfying consumers' taste for meat. With respect to sustainability, cellular agriculture can minimize the environmental impact dramatically, where equivalent land and water usage to produce animal tissue is 99% less than that of traditional animal agriculture methods. Additionally, cellular agriculture uses less total energy and produces less pollution than all conventional animal agriculture areas except poultry. For animal welfare, cellular agriculture has been recognized by PETA and other organizations as a means to eliminate the need for animal slaughter and dramatically minimize the amount of animal harm involved in meat production. In order to obtain initial cell samples, only small, harmless biopsies would be required to produce thousands of pounds of meat. Public health can also be improved using cellular agriculture because it is done in a sterile environment without the inherent risks of factory farming, and the meat produced can contain nutritionally beneficial compounds.

Dried meat products, such as beef jerky, were particularly found to be suitable for production as dried meat snacks are a $2.8 billion industry in the United States and are currently an untapped market within cellular agriculture. Further, the nature of dried meat snacks is that they are primarily made from the leanest cuts of meat, and thus, the cell culture process only requires muscle cells. One further advantage is that dried meat products rely less on the taste of the meat itself, as they are heavily flavored during processing.

It has been found herein that the decellularized plant tissues can be used as adaptable scaffolds for culture of animal cells, and particularly animal muscle cells, for production of edible animal tissue (e.g., dried meat products). Particularly, suitable scaffolds have large surface areas for cell attachment and growth. Further, effective scaffolds can maximize medium diffusion before the separation of cultured cells. Cells are surprisingly able to adhere to the scaffolds without the use of adherents and are further able to conform to the microstructure of the plant frameworks, resulting in cell alignment and pattern registration.

### Decellularized Plant Scaffolds

Generally, any plant tissue suitable for decellularization as known in the art is suitable as a source for plant tissue in the methods of the present disclosure. For example, the plant tissue can include leaf tissue, stem tissue, root tissue, seed, fruit, flower, and combinations thereof. Further, any plants known in the art can be used. Without being limiting, exemplary plants include spinach, leeks, iceburg lettuce, romaine lettuce, swiss chard, sweet wormwood, parsley, vanilla, and peanut, and combinations thereof.

Initially, the plant tissues are decellularized to eliminate compatibility issues. Particularly, the decellularization process allows for removal of cellular material from a tissue or organ leaving behind an acellular scaffold consisting of extracellular matrix (ECM), the composition of which depends on the tissue or organ from which it was derived (i.e., plant tissue), and can preserve an intact vascular network if desired.

Generally, the plant tissue is decellularized using any methods known in the art for decellularizing tissue. Generally, the plant tissue is decellularized via detergent perfusion using at least one of a detergent and enzyme. Exemplary perfusion methods include immersion in detergents and bleaching agents such as sodium hypochlorite (bleach), sodium dodecyl sulfate, sodium hydroxide, ethylenediaminetetraacetic acid (EDTA), Triton X-100, and the like, and combinations thereof. Exemplary enzymes for use in decellularization include lipases, thermolysin, galactosidases, nucleases (e.g., endonucleases such as benzoase), trypsin and combinations thereof. In some embodiments, the plant tissue can be decellularized using a mixture of detergent and enzyme, such as a mixture of EDTA and trypsin.

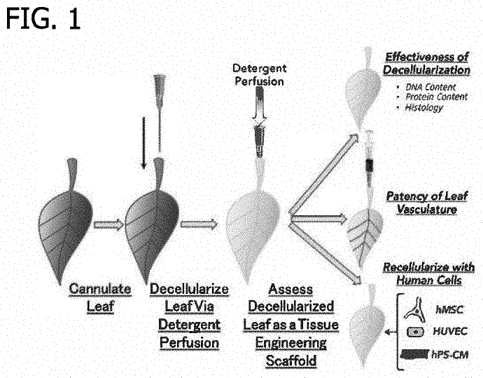

In particularly suitable embodiments, the decellularization process is a bulk decellularization process. The need for a bulk decellularizing system is due to the intensive user interfacing and non-scalability of the currently used leaf cannulation processes. For example, one design choice for the apparatus used in bulk decellularization for use in the methods of the present disclosure was a constant or intermittent flow system designed to perfuse several stages of detergents through the vasculature of plant leaves. The standard cannulation process involved suturing surgical needles into the stems of all the leaves, and washing them rigorously with hexanes (FIG. 1).

Another design for decellularization is to continuously stir or shake plant material in a container having an open upper end (e.g., beaker) and filled with different detergents and/or enzymes as discussed herein (for example, SDS-Tween 20+Bleach-DI H₂O-Tris Buffer). Typically, the plant material is soaked with the different detergents and/or enzymes for a time period of from about 30 minutes to about 72 hours. The materials can be stirred with any device known to be capable of mechanically stirring. For example, in one embodiment, the stirring device can include a stir bar, stir bar protector plate, and a stir plate.

Another method for large scale decellularization of plant tissue is the treatment of plant tissue sequentially with sodium hydroxide and sodium hypochlorite solutions, ranging in concentration from 2% to 40% by volume, in a vessel for 30 minutes to 72 hours typically. The containing vessel may be an open beaker, with or without stirring, or any of the apparatuses described herein.

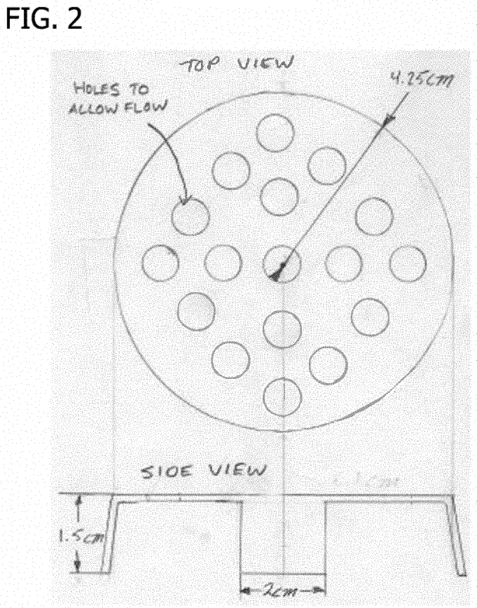

One alternative design used with continuous stirring includes an aluminum protective grate. The grate prevents the leaves from settling to the bottom of the beaker, prevents disruption of the stir bar, allows flow in the system, and protects the leaves from being damaged (FIG. 2).

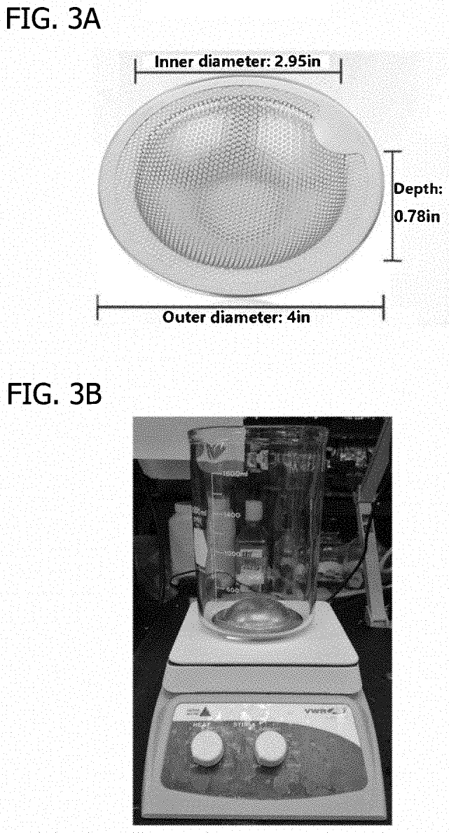

In one particularly suitable embodiment, an apparatus for decellularization is designed with the protective grate described above, but is altered to prevent the leaves from not only becoming damaged but clumping together on top of the protective grate. Particularly, a tiered grate for the leaves is used (FIG. 3A). The tiered grate decellularizing system (FIG. 3B) separates the leaves from each other. The tiers would run vertical so that the leaves are above each other, but would not come into contact with each other. The number of tiers may be altered as needed. In one particularly suitable embodiment, the tiered grate comprises at least two tiers, the first tier having a diameter smaller than the detergent vessel second tier. For example, the first tier can have a diameter being about 1 inch smaller than the second tier. Further, the tiered grate can include a depth ranging from 0.25 inches to about 4 inches, including about 0.78 inches.

Typically, the grate can be made of aluminum or stainless steel. Advantages of this embodiment include, for example, being scalable to industrial levels and being capable of consistently decellularizing various types of leaves. Its potential modularized design also allows for easy setup and removal of the plant leaves.

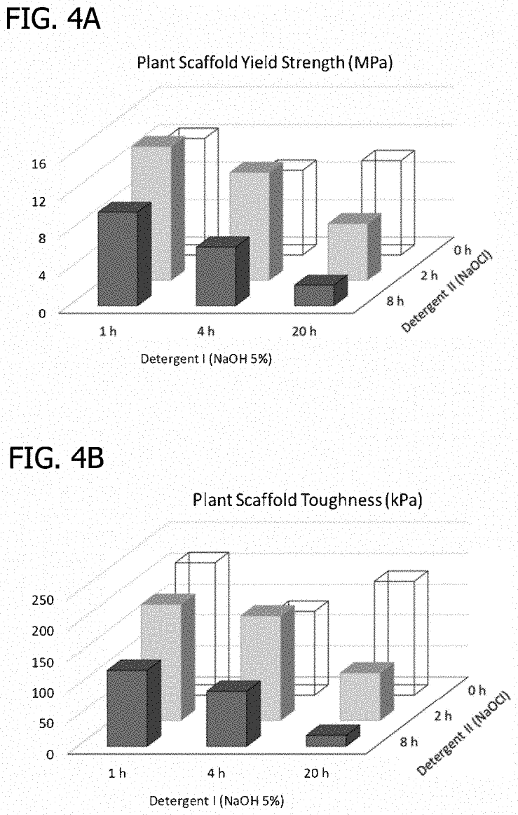

The decellularization process conditions, including but not limited to detergent concentration and treatment duration, may be altered to provide suitable mechanical properties of the plant scaffold. An example is shown in FIGS. 4A and 4B, where the duration of the sequential detergent steps is varied to provide a range of scaffold strength and toughness. Depending on the target meat product, the desired mechanical properties may be varied to a suitable range.

While the cells are found to adhere to the decellularized plant scaffolds without adhesion molecules, in some embodiments, the decellularized plant tissue can be functionalized to provide improved adhesion or other improved functioning (also referred to herein as “biofunctionalized”). In one embodiment, the decellularized plant tissue is functionalized by mineralization of the plant tissue. More particularly, the decellularized plant tissue is incubated in a modified simulated body fluid (mSBF) to form a mineral layer coating on the surface of the decellularized plant tissue. In some embodiments, the decellularized plant tissue is incubated in mSBF for a period of from about 7 to about 14 days with gentle agitation. Suitable mSBF contains a suitable mineral-forming material to form the mineral layer. Suitable mineral-forming materials may be, for example, calcium, phosphate, carbonate, and combinations thereof.

The modified simulated body fluid (mSBF) for use in forming the mineral layer typically included from about 5 mM to about 12.5 mM calcium ions, typically 2-12.5 mM phosphate ions, and 4-150 mM carbonate ions.

The resulting deposited mineral layer generally predominately includes calcium carbonate, phosphate, magnesium, and potassium. In some particularly suitable embodiments, the resulting mineral layer includes calcium and phosphate in a calcium to phosphate ratio from about 2.5:1 to about 1:1.

The pH of the resulting mineral layer may typically range from about 4 to 7.5, most typically 5.7 to 6.8.

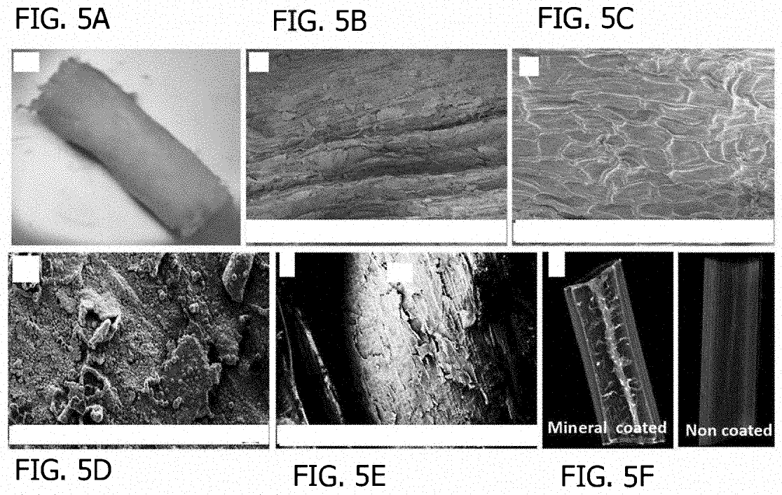

An example of mineralized plant scaffold is provided in FIGS. 5A-5F, where in micrographs of mineralized parsley stem and bamboo stem scaffolds are depicted and shown in comparison to the non-mineralized parsley stem and bamboo stem scaffolds.

In some embodiments, the mineral layer for mineralization of the decellularized plant tissue may further include a biomolecule that are suspected of binding or interacting with a cell to affect cell attachment, spreading, migration, maturation, expansion, proliferation, differentiation, and formation of cellular structures (e.g., tubules). Particularly suitable biomolecules can be nucleic acids, proteins, peptides, growth factors, proteoglycans, and combinations thereof. Suitable growth factors can be, for example, bone morphogenic protein, fibroblast growth factor, growth differentiation factor, platelet-derived growth factor, placental growth factor, transforming growth factor, insulin-like growth factor, vascular endothelial growth factor, bone sialoprotein, phosphoryn, osteonectin and combinations thereof. More particularly suitable growth factors can be, for example, vascular endothelial growth factor, bone morphogenetic proteins, fibroblast growth factor, insulin-like growth factor and combinations thereof. Suitable proteoglycans can be, for example, proteoglycans with heparin, heparin sulfate, and/or chondroitin glycosaminoglycan side chains.

In another embodiment, the decellularized plant tissue may be used without mineralization or further functionalization. The plant scaffolds may be rinsed with water after treatment with detergents and enzymes as described above.

Alternatively, the plant scaffolds is treated with buffers (including but not limited to Tris-hydrochloride, sodium phosphates, and citric acid), after the decellularization process. This step may or may not precede the functionalizations described above and below.

In another embodiment, the decellularized plant tissue is functionalized by decorating the decellularized plant tissue with adhesive cues such to allow adhesion of cells to the decellularized plant tissue. Particularly, the decellularized plant tissue can be contacted and/or coated with a plant adhesion molecule pre-conjugated to a cell adhesion peptide. Particularly, it was found that decellularized plant tissues that were coated with cell adhesion peptides pre-conjugated to plant adhesion molecules allowed for effective cell adhesion, even enabling human cell adhesion on plant tissues.

Suitable plant adhesion molecules include dopamine-containing compounds (including polydopamines), polyphenols and combinations thereof. Dopamine is a catechol moiety found in adhesive proteins and is capable of strong adhesion in aqueous environments. Without being limiting, exemplary dopamine-containing compounds include dopamine hydrochloride.

The plant adhesion protein is conjugated with a cell adhesive peptide prior to coating the decellularized plant tissue. As used herein, a “cell adhesion peptide” refers to an amino acid sequence obtained from an adhesion protein to which cells bind via a receptor-ligand interaction. Varying the cell adhesion peptide and concentrations thereof in the solution allow for the ability to control the stability of the cellular attachment to the resulting functionalized, decellularized plant scaffold. Suitable cell adhesion peptides include, for example, RGD, RGDS (SEQ ID NO:1), CRGDS (SEQ ID NO:2), CRGDSP (SEQ ID NO:3), PHSRN (SEQ ID NO:4), GWGGRGDSP (SEQ ID NO:5), SIDQVEPYSSTAQ (SEQ ID NO:6), GRNIAEIIKDI (SEQ ID NO:7), DITYVRLKF (SEQ ID NO:8), DITVTLNRL (SEQ ID NO:9), GRYVVLPR (SEQ ID NO:10), GNRWHSIYITRFG (SEQ ID NO:11), GASIKVAVSADR (SEQ ID NO:12), GTTVKYIFR (SEQ ID NO:13), GSIKIRGTYS (SEQ ID NO:14), GSINNNR (SEQ ID NO:15), SDPGYIGSR (SEQ ID NO:16), YIGSR (SEQ ID NO:17), GTPGPQGIAGQGVV (SEQ ID NO:18), GTPGPQGIAGQRVV (SEQ ID NO:19), MNYYSNS (SEQ ID NO:20), KKQRFRHRNRKG (SEQ ID NO:21), CRGDGGGGGGGGGGGGGPHSRN (SEQ ID NO:22), CPHSRNSGSGSGSGSGRGD (SEQ ID NO:23), Acetylated-GCYGRGDSPG (SEQ ID NO:24), CRDGS (SEQ ID NO:25), cyclic RGD{Fd}C (SEQ ID NO:26), RKRLQVQLSIRT (SEQ ID NO:27), IKVAV (SEQ ID NO:28), YIGSR (SEQ ID NO:29), KRTGQYKL (SEQ ID NO:30), TYRSRKY (SEQ ID NO:31), KRTGQYKLGSKTGPGQK (SEQ ID NO:32), QAKHKQRKRLKSSC (SEQ ID NO:33), SPKHHSQRARKKKNKNC (SEQ ID NO:34), XBBXBX, wherein B=basic residue and X=hydropathic residue (SEQ ID NO:35), XBBBXXBX, wherein B=basic residue and X=hydropathic residue (SEQ ID NO:36), and RGDSP (SEQ ID NO:37).

The present disclosure further may include a spacer peptide between the plant adhesion molecule and cell adhesion peptide. The addition of a spacer in the peptide sequence ensures that the conjugation with the plant adhesion molecule (e.g., dopamine-containing compound) does not affect the bioavailability of the cell adhesion peptide. Suitable spacer peptides for use herein include, for example, poly-glycine or glycine-rich sequences (e.g., GGG, GSGSGS (SEQ ID NO:38), etc.)

To aid in conjugation, cross-linking agents are used. Suitable cross-linking agents include, for example, 1-Ethyl-3-(3-dimethylaminopropyl)-carbodiimide (EDC) and N-hydroxysuccinimide (NHS), aldehydes (e.g., glutaraldehyde), isocyanates, plant extracts, and the like and combinations thereof.

The concentration of conjugated plant adhesion molecule and cell adhesion peptide for coating the decellularized plant tissue will depend on the specific cell adhesion peptide being used and the desired cells to be adhered to the decelluarized plant tissue. Typically, however, the decellularized plant tissue is coated with from about 0.1 mg/mL to about 1 mg/mL conjugated plant adhesion molecule and cell adhesion peptide.

The plant scaffolds of the present disclosure can be used to alter (e.g., enhance, inhibit and change) cell function, and in particular, cellular expansion, maturation and differentiation. Cells can be analyzed for cell attachment, cell spreading, cell morphology, cell proliferation, cell migration, cell expansion, cell differentiation, protein expression, cell-to-cell contact formation, sprouting, tubulogenesis, formation of structures, and combinations thereof.

### Uses of Plant Scaffolds

The present disclosure is directed to preparing edible animal tissues using the decellularized plant scaffolds described above. Generally, the processes begin by decellularizing the plant tissues as discussed above to form decellularlized plant scaffolds. Cells are then seeded onto the surface of the decellularlized plant scaffolds.

Use of plant scaffolds is described below using the specific example of “leaves”. However, in all cases, “leaves” may be substituted with other plant scaffolds, including but not limited to scaffolds derived from root tissues, stems, leaflets, seeds, fruits, flower, and combinations thereof.

The method of seeding can be any method known in the art. Exemplary methods for seeding the cells to the decellularized plant scaffolds include spraying the leaves, coating the leaves, submersing the leaves in cell media, and perfusing cell media throughout the leafs vasculature. It should be noted that any cell media known in the seeding art may be used without departing from the scope of the present disclosure.

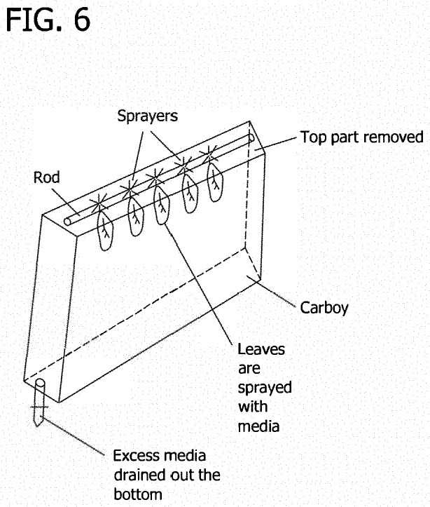

More particularly, in one embodiment, seeding of the cells incorporates the use of a carboy and hanging the leaves. The top of the carboy is removed and the leaves are hung along a vertical rod across the box. The leaves will be seeded with cells, and instead of putting them in cell media directly they are spritzed with media. The leaves would be sprayed intermittently for a period until the desired seeding is accomplished, for example, the leaves could be sprayed once, twice, three times, four times or more an hour for a period at a few days to a month or so to allow a thick tissue to form on the leafs surface. Both sides of the leaf can be seeded to encourage formation of tissue on both sides. FIG. 6 depicts one design for this embodiment.

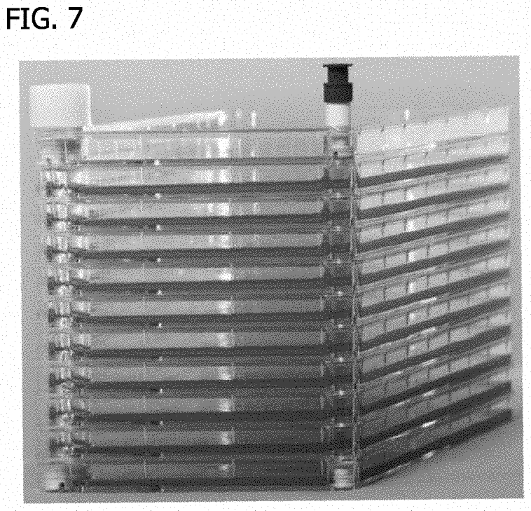

In another embodiment, the leaves are placed in a cell media bath. For example, a system such as a ThermoFisher Scientific Nunc™ EasyFill™ Cell Factory™ System or the like is used. This type of system helps to maximize the amount of laboratory space when trying to grow animal tissue on leaves. Each layer would contain one, or possibly more, seeded leaves bathed in cell media. Media is added and removed from the top of the system and can be equally distributed between all layers. FIG. 7 depicts such a system.

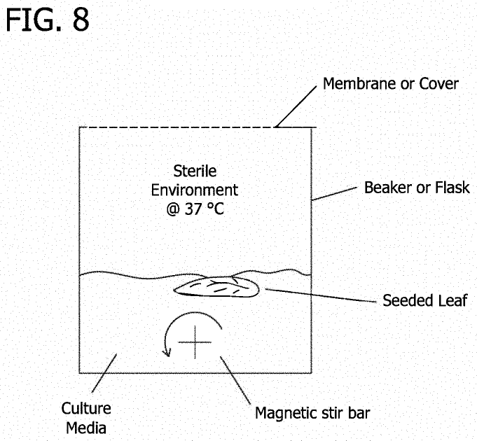

Yet in another embodiment, cells are seeded by placing seeded leaves in a media bath that is agitated by a magnetic stir bar. The agitation may provide a few possible benefits to the cells: 1) additional oxygenation, 2) increased perfusion of cell media throughout the leaf, 3) increased shear forces that could stimulate the leaf and drive the growth and differentiation of cells. FIG. 8 depicts such a system.

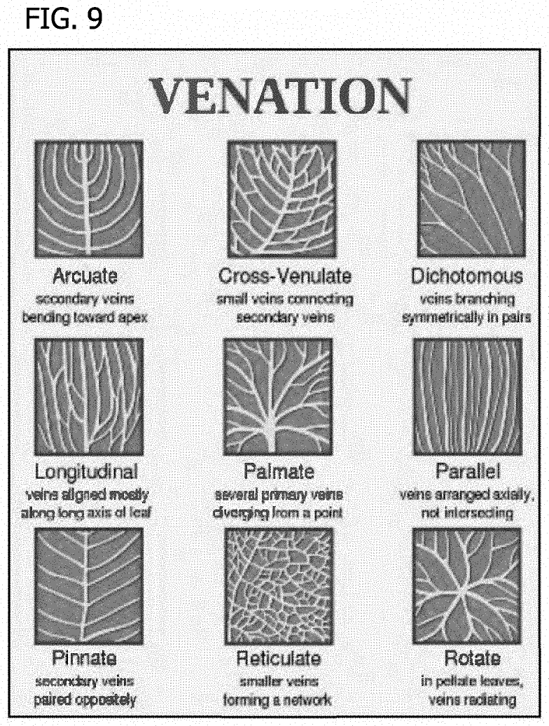

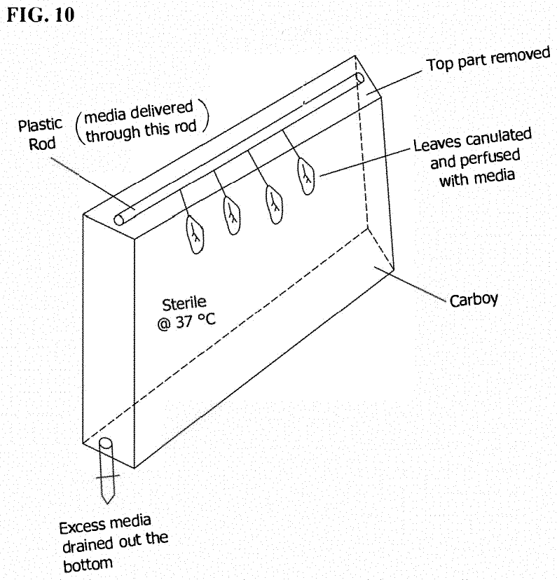

As shown in FIG. 10, in one embodiment, seeding involves the use of a carboy similar to that shown in FIG. 6, but instead of spraying the cells with culture media, the media is perfused throughout the leaf's vasculature. This design allows for maximized cell viability and growth by providing nutrients in a more efficient manner. An exemplary type of leaf venation system used is shown in FIG. 9.

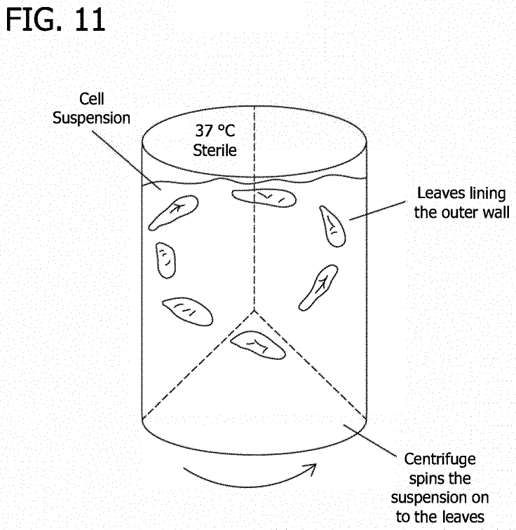

In one more suitable embodiment, a centrifuge can be used to deliver cells. Particularly, the leaves would be placed in a centrifuge along with a cell suspension. The leaves would be lined along the outer edge of the centrifuge. Once it spins, the cells will be driven along the centrifuge to guide their attachment to the cells. One alternative design can be used where cells are shot out of the center of the centrifuge and are guided to attach to the leaves lining the outer wall. FIG. 11 depicts this alternative design.

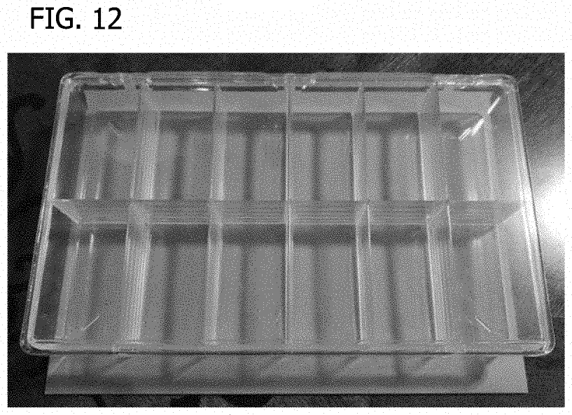

In yet one more suitable embodiment, an incubator “tackle box” is used for seeding. The incubation “tackle box” design is made from any polymer known in the art (e.g., polystyrene) to allow the incubator to be gamma irradiation, autoclaving, and ethylene oxide (EtO) sterilizable (ISM, 2018). The base of the incubator is a compartmented container with dimensions desirable for the form factor of a dried meat product (FIG. 12).

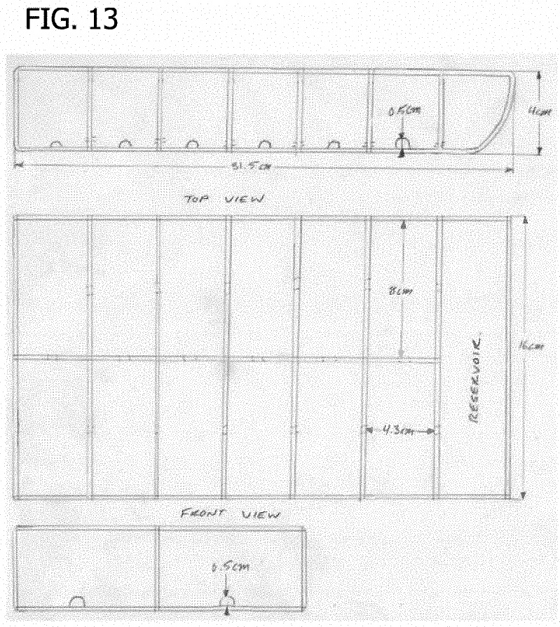

Leaves will be placed into each of the compartments of the incubator for initial seeding and proliferation. The dividing sections are perforated with small holes to allow for the equal exchange and leveling of media between compartments. A reservoir is attached to the lengthwise portion of the incubator, where media can be aspirated and added by tilting the box and allowing gravity to pool into the reservoir (FIG. 13).

This incubation “tackle box” design eliminates the potential of damaging the leaves during media exchange, and can be used for seeding and reseeding. The portable design and form of the box allows for it to be placed from a biosafety cabinet into an incubator. The design is fabricated to prevent airflow exchange into the compartments of the box to prevent contamination during transfer to and from biosafety cabinets and during incubation.

Suitable cells for seeding include myoblasts, myoblast progenitors, fibroblasts, adipocytes, adipocyte progenitors, osteoblasts, osteoblast progenitors, and combinations thereof. The cells are typically of animal origin, such as from bovine, pig, chicken, fish and the like.

### EXAMPLES

The following general materials and methods were used in Examples 1-7.

1. Preparation of Samples/Cells

It should be understood that the all procedures in these Examples should be performed using known laboratory methods, which reduce contamination of the meat sample as much as possible. The meat sample was first placed in a soaking medium, which contained antibiotics to help kill any surface contaminants. The soaking medium was made using 49.5 mL of F12 DMEM and 0.5 mL of Pen Strep.

Tissue digestion medium (DMEM/F12 (Ham's), 1% Pen Strep, 10% Collagenase Solution) was used to break down the collagen in the meat sample biopsies, which allowed cells to be more effectively isolated. Tissue digestion medium was made using 5 mL of the collagenase type I solution, prepared by adding collagenase type I to 5 mL of Hank's Balanced Salt Solution (1800 units/mL solution in HBSS), 0.5 mL of Pen Strep, and 44.5 mL of F12 DMEM.

Tissue rinse medium (Tissue Rinse Medium (DMEM/F12 (Ham's), 1% Pen Strep, 10% Fetal Bovine Serum)) was prepared for use during the filtering steps of the isolation. Tissue rinse medium was made using 5 mL of heat-inactivated FBS, 0.5 mL of Pen Strep, and 44.5 mL of F12 DMEM.

Cell Culture Growth Medium (DMEM/F12 (Ham's), 1% Pen Strep, 10% Fetal Bovine Serum, 4 ng/mL FGF2, 10 ng/mL EGF, 2.5 ng/mL, HGF, 5 ng/mL IGF1) for culturing the isolated cells was placed into a 500 mL F12 DMEM bottle. 55 mL of DMEM was removed from the bottle. 5 mL of Pen Strep, 50 mL of heat-inactivated FBS, and four pre-aliquoted growth factors were added to the DMEM bottle. The growth factors included: FGF2 (all 20 μl), IGF (all 25 μl), HGF (only 3.1 μl), and EGF (all 50 μl).

2. Isolation of Cells

A muscle sample was placed in a petri dish filled with 50 mL of soaking medium, allowed to soak for 10 minutes (turning over after 5 minutes). 10-20 interior penny-sized muscle biopsies were removed from the meat sample and placed in a petri dish filled with 50 mL of digestion medium.

The digestion medium dish was then moved into a 5% CO₂ incubator and incubated for 1 hour at 37° C. The dish was swirled every 15 minutes. After 1 hour, the contents of the disk were transferred into a 50 mL conical tube. Further, after letting the larger pieces settle, the small tissue pieces and medium (supernatant) were transferred through a 100 μm cell strainer into a new 50 mL conical tube where the contents were centrifuged for 5 minutes at 0.3 rcf.

The supernatant was aspirated with 5 mL of tissue rinse medium and the cell pellet gently titrated until the pellet was resuspended. The suspension was passed through a 70 μm cell strainer and transferred to a new conical tube. The spin/rinse/strain process was then repeated 3 times using a 40 μm cell strainer. After the third centrifugation, the cell pellet was resuspended in cell culture growth medium.

The cell suspension was transferred into a T-75 flask (10-12 mL of volume) and put in the incubator. The cell culture growth medium was changed every 2 days and passaged when the medium approached 70% confluency.

The isolation contained a mixture of fibroblasts, myoblasts, myosatellite cells, and, even muscle chunks that might not have been strained. It is extremely important for the isolation to include sufficient myoblasts and myosatellite cells in the culture, as these cell types will eventually differentiate into myocytes via contact with each other or the removal of growth factors from the media.

3. Culturing of Cells

After spinning down the cells and resuspending the cell pellet, there was two conical tubes: one that contained the cells that were originally floating in the media, and another than contained the cells that were trypsinized and adhered.

Plating Isolation Cells After Passaging:

The contents of both conical tubes are combined and growth factor media added until the total volume was 12 mL. 2 mL of the suspension was added to each well of the well plate, and the plate was placed in the incubator for five hours to allow the fibroblasts to attach to the bottom of the plate.

After 5 hours, the floating cells from each well were removed and plated in an appropriately sized flask. T-150 flasks are especially useful because they can hold double the volume of a T-75 and the cell population takes longer to become confluent. T-75 flasks typically can be seeded with 500 k cells, while T-150 flasks can be seeded with over a million cells.

The adhered cells are trypsinized again, and the process of passaging repeated.

In this case the tissue chunks should be treated separately.

Tissue Chunks Procedure:

For any tissue chunks from the well plate, the chunks were removed using a 10 mL pipette and a pipettor and transferred to a conical tube. The tissue chunks were spun down for 5 minutes at 0.3 rcf, aspirated out the media, and resuspended in 10 mL of trypsin. A micropipette was used to gently separate the chunks mechanically.

The tissue chunks were transferred to a T-75 flask and placed in the incubator for 15 minutes. The flask was agitated every 3 minutes.

Finally, the suspension was spun down, the trypsin aspirated, and the suspension was resuspended in growth factor media. After 15 minutes, the process of spinning/aspirating/resuspending in trypsin could be repeated again, if necessary. The suspension was then plated on an appropriately sized flask.

4. Seeding: Satellite Cells, Myoblasts and Fibroblasts onto Decelled Leaves

Plant Bulk Decellularization:

Leaves and stems were bulk decellularized. The plant leaves with first washed wiht distilled water. A stir bar and a stir bar protector plate were placed at the bottom of a 2 L beaker. The beaker was then filled with SDS to the 1000 mL mark. 10 plant leaves were placed into the beaker. Depending on the amount of leaves and the type of stir plate, set the stir plate to an appropriate rpm. The rpm should be set so the leaves are moving around, but are not being destroyed by the flow.

The leaves were allowed to soak in SDS for 24 hours. After 24 hours, the SDS was replaced with Triton X-100 or Tween 20+Bleach. After 24 hours, the Triton X-100/Tween 20+Bleach was replaced with DI H2O.

After 24 hours, r the DI H₂O was replaced with Tris Buffer. After 24 hours in Tris Buffer, the leaves were removed and frozen overnight in a −20° C. freezer. The leaves can stay in the freezer up to three weeks.

The leaves were lyophilized for 24 hours. Finally, the lyophilized leaf scaffold was stored at room temperature until needed. It should be understood that the rate of decellularization of the plants could be altered in numerous ways, for example, increasing the concentration of the decellularization chemicals could increase the rate of decellularization; increasing the stirring speed of the stir bar may increase the rate of decellularization; and adding fewer leaves to the vat could increase the rate of decellularization.

Preparing and rehydrating the decellurized leaves:

The decellurized leaves were first cut into the desired shape and sized and placed into a cell culture plate. The leaves were then covered in tris buffer solution and left for 30 minutes on a shaker plate. The tris buffer solution was aspirated and replaced with DI water and left for 30 minutes on a shaker plate.

The DI water was aspirated and replaced with 70% ethanol, and then left for 30 minutes. The plate was then rinsed with sterile PBS three times, waiting five minutes between each rinse. The leaves were moved into a sanitized polystyrene container that fits the shapes of the leaves. The leaves were covered in cell growth media and incubated overnight.

Seeding cells onto leaves:

Passage and count your cell supply

The cells were passaged as described above and cells counted using the following procedure: 10 ul of cell+trypan blue mixture was loaded in each side of a hemocytometer. Boxes were counted to achieve a count of 100 cells of greater. The formula to determine the cell density is:

# of cells counted# of boxes counted*2*10,000*# of ml=cell countl ml

The desired amount, which typically ranges from 200 k to 300 k cells per cm² of decellularized leaf surface area, but can be as low as 5 k/cm², of cells were deposited onto each leaf, and sufficient amount of growth media to cover the leaf was deposited. The plates were incubated. The media was checked daily and refeed every other day.

5. Analysis of Plant Scaffolds

Phalloidin/Hoechst Staining

The reagents used included: Phosphate Buffered Saline; 4% Paraformaldehyde (Only needed for tissues/cells that have not been fixed); 0.25% Triton-X; 0.25% V/V Triton-X in PBS; 10 μL Triton-X in 3990 μL PBS; 1% BSA; 1% V (W)/V BSA in PBS; 40 μL in 3960 μL PBS; Phalloidin (AF 488 Phalloidin A12379 or FITC Phalloidin, Invitrogen); 2.5% V/V Phalloidin in PBS; 50 μL in 1950 μL; Hoechst: 0.0167% Hoechst dye in PBS; 0.5 μL in 3000 μL PBS.

For unfixed sections/cells: the cells were first rinsed in PBS ×2 and then fixed in 4% Paraformaldehyde for 10 minutes. The cells were then again rinsed in PBS ×2, and then the procedure for fixed cells was followed.

For fixed sections/cells, the cells were rinsed with PBS ×2 and Triton-X solution for 10 minutes. The cells were then again rinsed with PBS ×2 and blocked with BSA solution for 30 minutes. The cells were put into the Phalloidin solution for 30 minutes, and then rinsed again with PBS ×2.

The cells were put into the Hoechst solution for 3-5 minutes and rinsed with PBS ×2. The cells were optionally cytosealed and a coverslip was used to cover the plates. The plates were stored frozen at −20° C.

F-actin would be stained green if 488 was used, red if FITC was used, and the nucleus would be stained blue.

MF20 Staining (Myocyte Staining)

The reagents used included: 5% Normal Goat Serum; Primary mouse monoclonal MF20 in 5% goat serum (1:30); Secondary antibody-goat anti-mouse Alexa Fluor 488 in 5% goat serum (1:400); Hoescht—0.0167% Hoescht dye in PBS, 0.5 uL in 3,000 uL PBS.

For fixed tissue samples, the tissue was thawed in PBS for 5 minutes, and then placed into 0.25% Triton-X-100 for 10 minutes. Then, the samples were washed in PBS for 5 minutes and the wash was repeated 3 times. The reaction was blocked with 5% Normal Goat Serum for 45 minutes (the goat serum was left on negatives but aspirated off the positives).

Primary mouse monoclonal anti-myosin was added for 1 hour at room temperature and then the samples were washed in PBS for 5 minutes and the wash was repeated 3 times.

Secondary antibody goat anti-mouse Alexa Fluor 488 was contacted with the samples for 1 hour at room temperature in the dark, and again, the samples were washed in PBS for 5 minutes and the wash was repeated 3 times.

The tissue samples were then contacted with Hoescht solution—1:6000 in PBS for 5 minutes and washed in PBS for 5 minutes, the wash repeated 3 times.

The samples were cytosealed and stored frozen in −20° C. MF20: green; Nuclei: blue.

### Example 1

In Examples 1 and 2, bulk decellularization was carried out using the beaker and stir bar method.

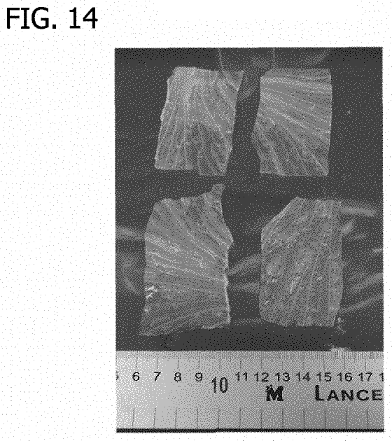

In Example 1, a bulk batch of iceberg lettuce was decellularized as described above. Leaves in the decellularization process are shown in FIG. 14.

The goal of the SDS step was to wash away oils or contaminants on the surface of the leaf. The Triton-X+Bleach step washed away all of the cells and chloroplasts, leaving behind a clear cellulose backbone. The D.I. H₂O and Tris Buffer steps were used to wash out the excess SDS, Bleach, and Triton-X before the leaves are lyophilized, rehydrated, and seeded with cells.

### Example 2

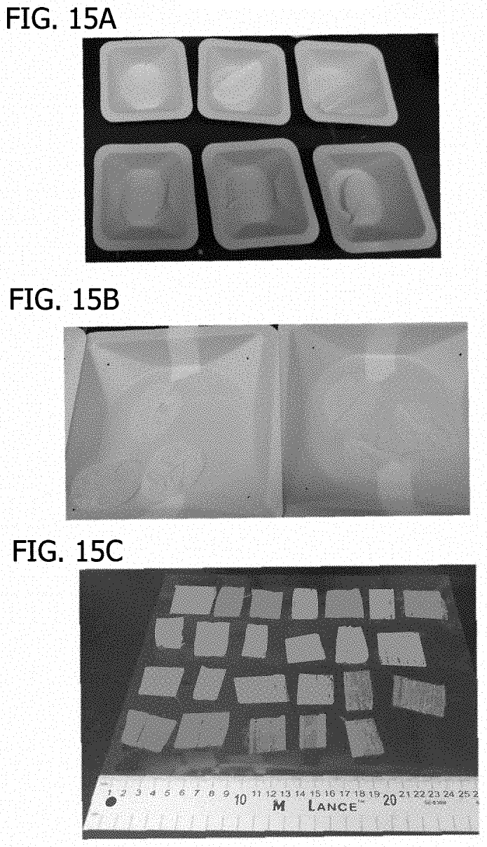

In this Example, bulk decellularization of spinach leaves was carried out using the aluminum protective plate cut with circular holes as discussed herein. Tween 20 was substituted for Triton-X as used in Example 1 (FIGS. 15A & 15B).

Further, bulk decellularization of leek leaves was also carried out using the same procedures. Leaves in the decellularization process are shown in FIG. 15C.

### Example 3

In this Example, P7 isolated cells from cow muscle were seeded onto a 24-well plate containing 12-wells of decellularized and lyophilized spinach and 12 wells of decellularized and lyophilized iceberg lettuce leaves. Leaves were seeded at an initial density of 200 k cells per construct using pyrex cloning wells and were left to incubate for 4 days without the removal of growth factors. After 4 days of incubation, cells were fixed and stained used phalloidin-actin alexa fluor 488 and hoechst 33342. Cells were then imaged under a fluorescent microscope.

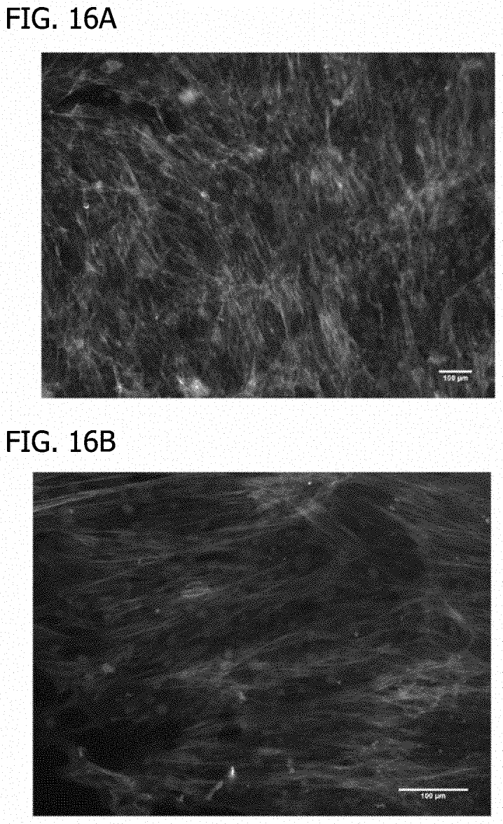

The spinach showed a confluent monolayer of cells spread along the top of the of spinach leaves, with the green coloring represented actin, and the blue representing nuclei (FIGS. 16A & 16B).

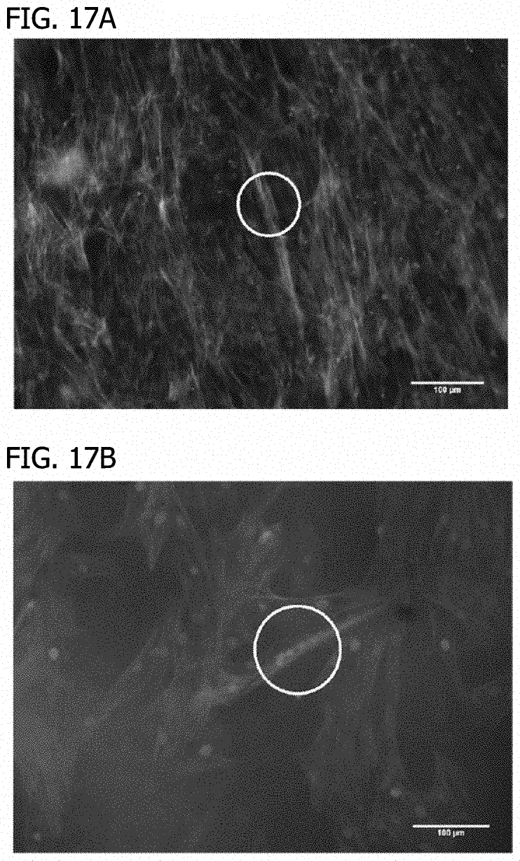

In addition to the confluency observed on the cells, there was evidence of multinucleation and myocyte development on the leaves as well (FIGS. 17A & 17B).

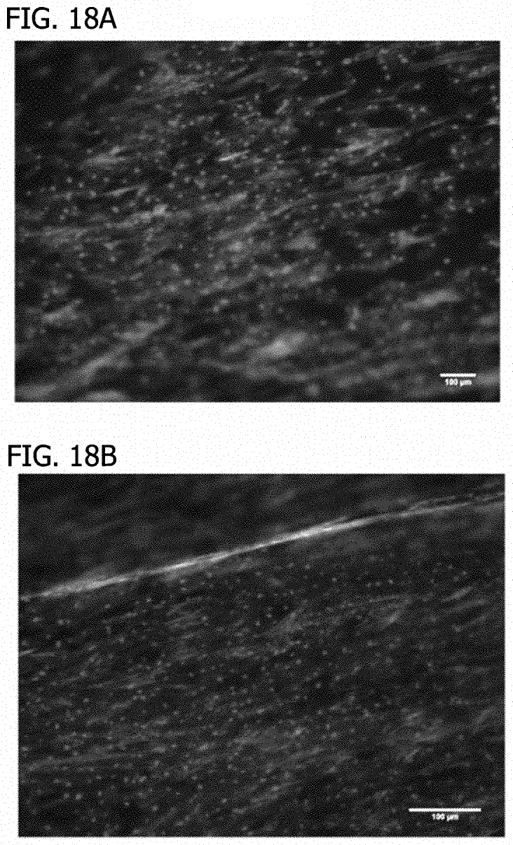

The evidence of this monolayer of cells was consistent with the imaged iceberg lettuce leaves as well (FIGS. 18A & 18B).

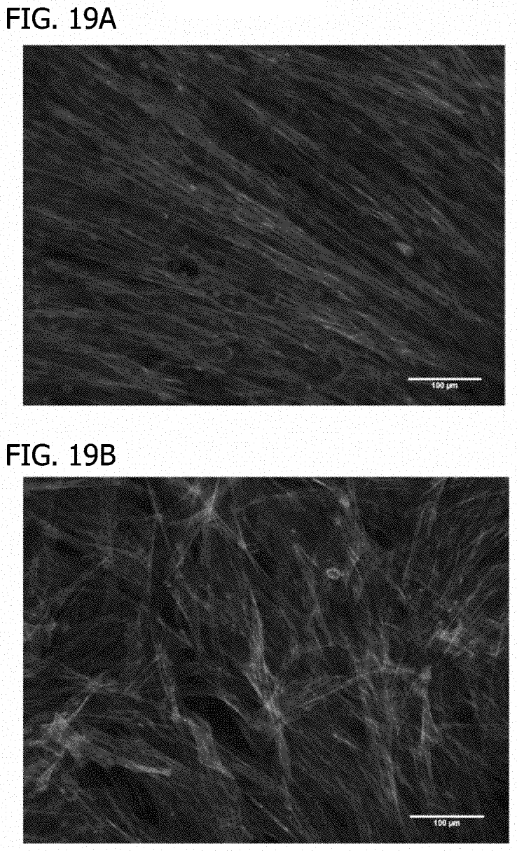

There was some evidence of alignment and striation of the muscle in the figures. However, since these samples were not stained for MF-20 for heavy chain myosin, the actual extent of differentiation and muscle striation was only speculative. The resulting images were conclusive with a triplicate control well that was seeded and incubated in parallel with the leaf samples (FIGS. 19A & 19B).

The results of the experiments showed successful proliferation of muscle skeletal bovine cells in a monolayer on top of the decellularized leaves. Additionally, evidence of differentiation and multinucleated cells occurring was visible.

### Example 4

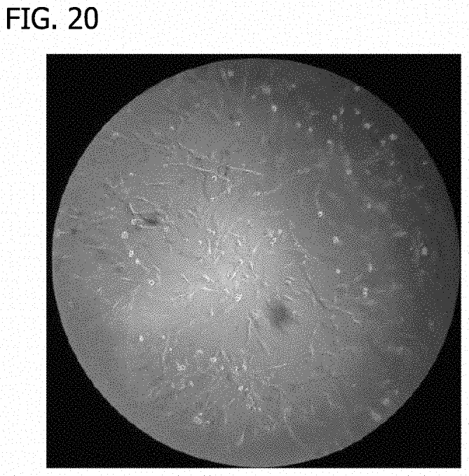

In this Example, thawed isolated myoblasts (P8) were passaged and seeded in spinach leaves (FIG. 20). In this Example, approximately 400 k cells were seeded per well on a well plate. The cells were allowed to grow for four days with growth factor media. The cells were then fixed and stained according to the MF20/Hoechst staining protocol above.

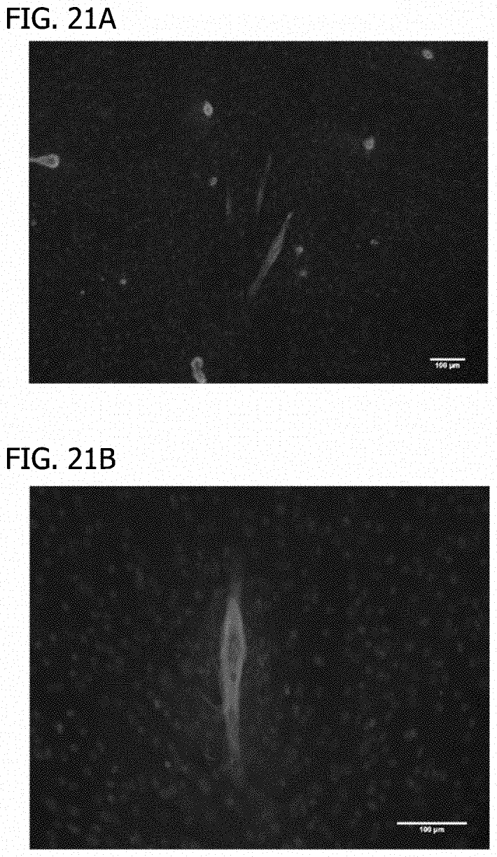

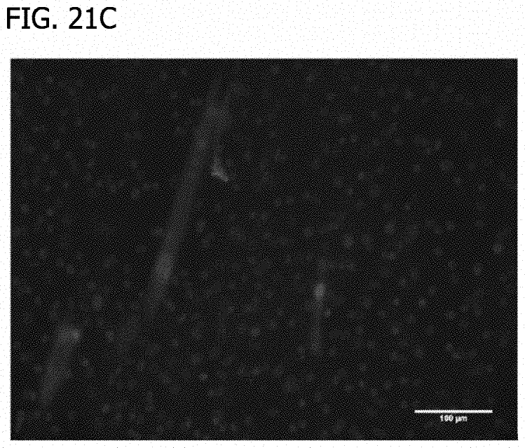

There was some evidence that some myoblasts were differentiating due to contact with other myoblasts in the wells. The green myosin heavy chain stain shows multiple areas in which there are multinucleated myocytes (FIGS. 21A-21C).

### Example 5

In this Example, approximately 130 k myoblasts were seeded per construct on a 24-well plate. There were 5 constructs with spinach leaf portions in row C and there were 6 constructs with spinach leaf portions in row D. Row C was designated for cells that received media with growth factors for the entirety of the experiment (9 days). Row D was designated for cells that received media with growth factors for four days and then media without growth factors for five days. The removal of growth factors is supposed to encourage myoblast fusion and myocyte (muscle fiber) formation.

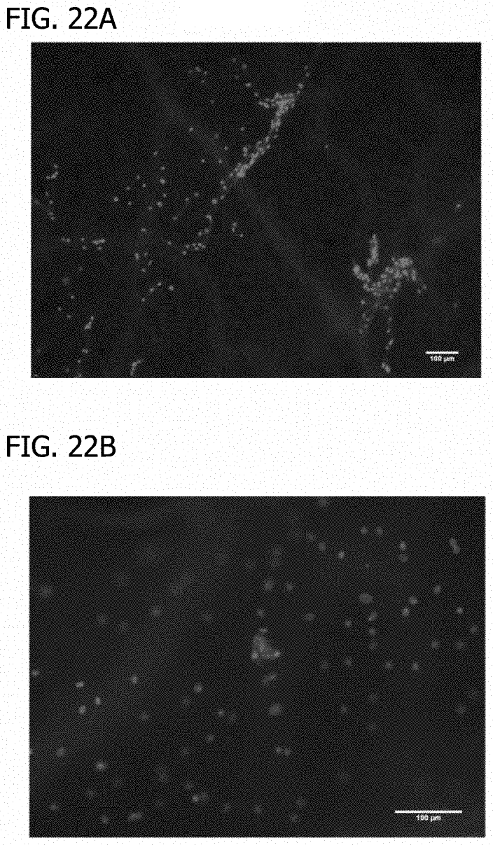

FIG. 23 shows the cells on the D row leaves without the use of a primary antibody. Therefore, only the Hoescht staining is visible. There is no fluorescence from the secondary antibody. This shows that none of it attached to unwanted areas.

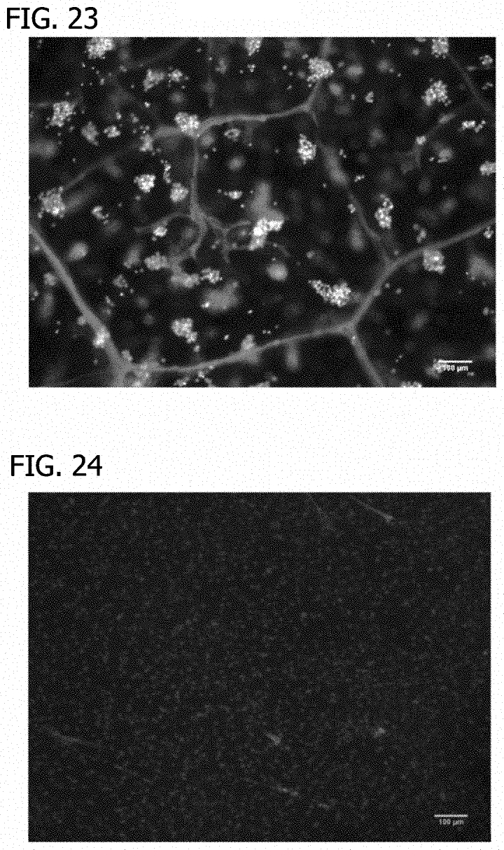

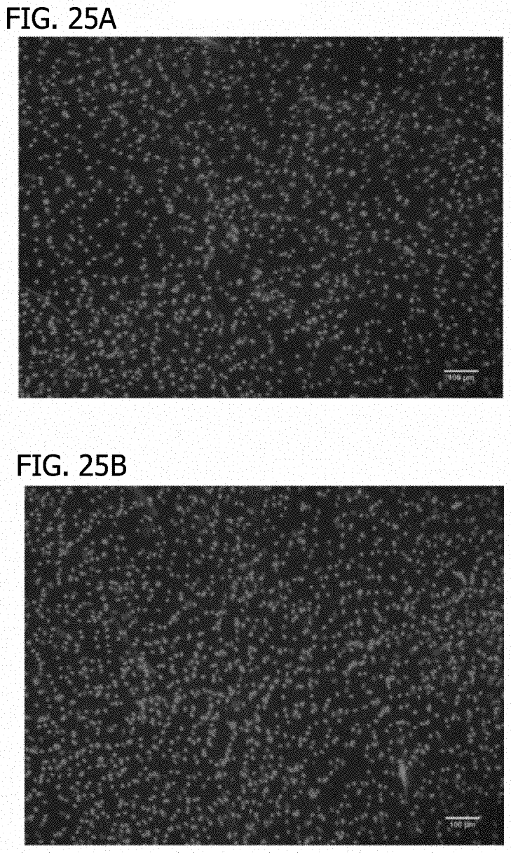

FIGS. 24 & 25A & 25B show the control wells without leaves. There were a few green lines, possibly representing myocytes. The wells following the protocol of row D (FIGS. 25A & 25B) seemed to be more confluent than the well that followed the protocol of row C (FIG. 24). The difference, however, is not high. It is unknown whether replacing the growth media with differentiation media yielded adequate results.

### Example 6

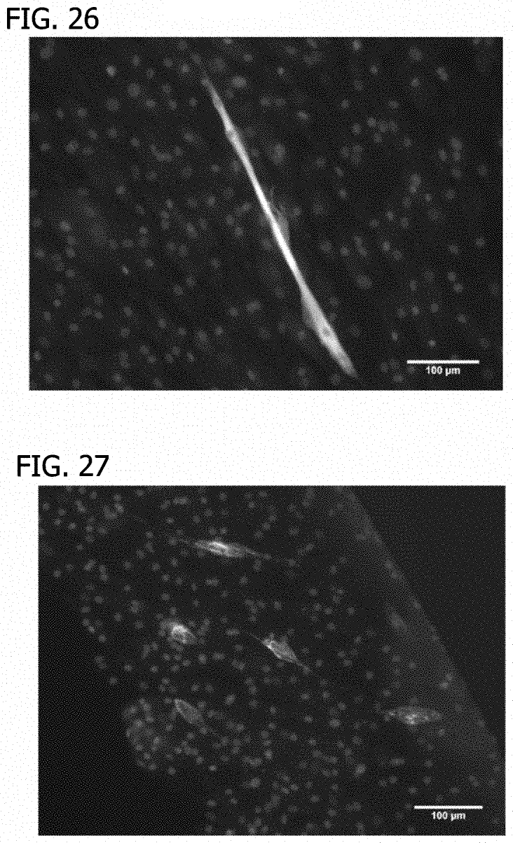

In this Example, three different differentiation focused experiments were conducted in parallel using the same cells. The first of the three experiments was using growth factor media exclusively for a 9-day period, replacing media every 2 days. After 9 days, the cells were stained with MF-20 and Hoescht 33342 to observe the presence of differentiated myoblasts attached to the lettuce (FIG. 26).

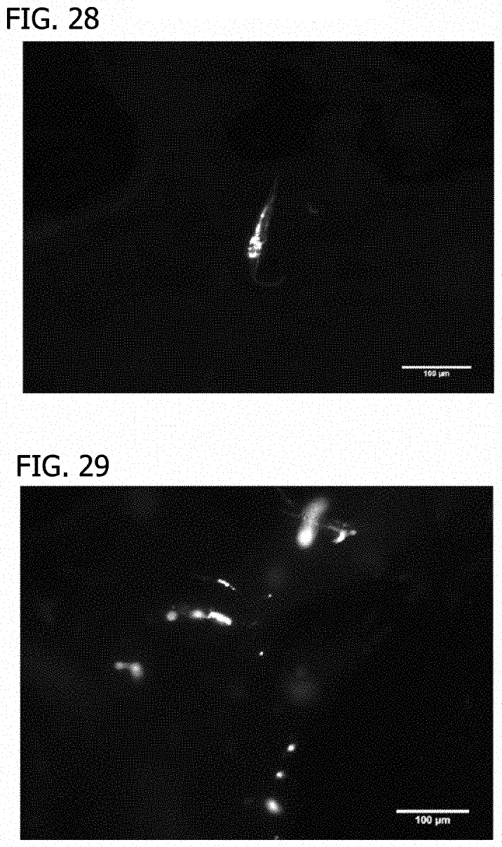

The second experiment was changed to non-growth factor media on the 4^{th} day, and were also cultured for nine days total. After 9 days, the wells were stained with MF-20 and Hoescht 33342 to examine the effects of differentiation (FIG. 27). The green markers show multiple areas of differentiation beginning on the surface of the leaf.

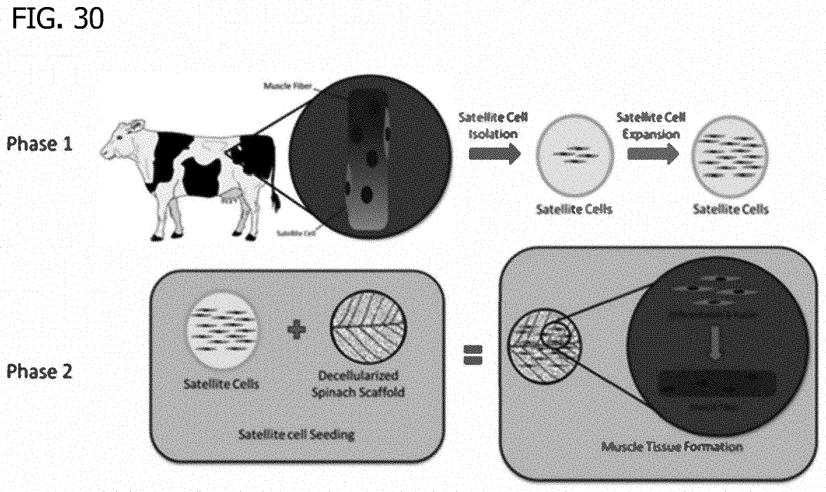

The third and final experiment used growth factor media for 4 days, and was then switched to non-growth factor media for a total culture time of 18 days. Approximately 200 k (PASSAGE 10) cells were seeded onto lettuce in a 24-well plate. The MF-20 markers were still observed on the lettuce, but compared to the previous two experiments no significant difference was observed (FIG. 28).

### Example 7

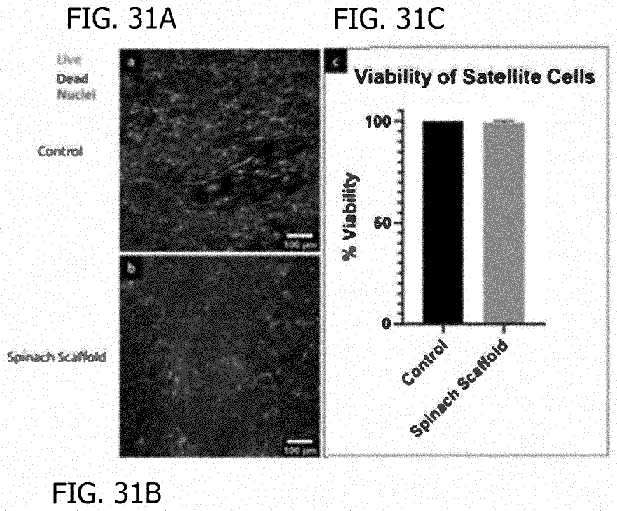

In the following Example, two different tests for seeding efficacy were conducted. PASSAGE 10 cells were seeded at a density of approximately 200 k into six different wells of a 24-well plate using cloning wells for each of the two experiments. The first experiment used growth factor media exclusively for 18 days, reseeding on every 5^{th} day. After the 18 days, the cells were stained with MF-20 and Hoescht 33342 (FIG. 29). The nuclei overlay was omitted in this picture due to the high autofluorescence caused by multiple layers of nuclei present in the fibroblast cells.

In the second experiment, cells were seeded and incubated for 4 days with growth factor media, and then changed to non-growth factor media for 5 days. Cells were then seeded again, and media was replaced with growth factor media for 4 days, and then replaced with non-growth factor media for 5 days. After 18 days, or two cycles, the cells were fixed and stained using MF-20 and Hoescht 33342. During imaging it was observed the most of the cell layers had sheared off, likely due to a combination of aspirating, handling, and layers shearing off with the cloning wells.

### Example 8

In this Example, decellularized spinach for use as a scaffold for in lab-grown meat applications was analyzed for viability, differentiation potential, and relative alignment of seeded bovine primary satellite cells.

Each experiment was done with 3 biological replicates with cells isolated from three different cows grown on decellularized spinach. These biological replicates were referred to as cow 1, cow 2, and cow 3. Each biological replicate had 3 technical replicates for a total N of 9. These samples were compared to a control group of isolated satellite cells grown on gelatin coated glass slides.

Spinach Leaf Decellularization and Scaffold Preparation

Baby spinach leaves were acquired from a grocery store. Spinach cuticles were removed through cyclically agitating the leaves in 98% hexanes (VWR, Radnor, Pa.) for 3 minutes followed by Phosphate Buffered Saline (PBS) for 3 minutes. Cuticle removal was achieved after 3 cycles of hexanes and PBS treatment. After complete cuticle removal, spinach leaves were placed in 50 ml conical tubes and submerged in 1% sodium dodecyl sulfate (SDS) (Sigma-Aldrich, St. Louis, Mo.) in deionized water for 5 days, refreshing the solution. After the initial 5 days, the SDS solution was replaced with 0.1% Triton X-100 (Sigma-Aldrich, St. Louis, Mo.) and 10% concentrated bleach in deionized water for 48 hours, refreshing the solution. The spinach leaves were then rinsed in deionized water for 24 hours. Following rinsing, the leaves were placed in 10 mM tris buffer (Sigma-Aldrich, St. Louis, Mo.) for 24 hours. The leaves were stored at −20° C. overnight. Lyophilization (FreeZone Triad 74000 series) was performed at −25° C. and 0.210 Torr over 24 hours. Decellularized spinach scaffolds were stored at room temperature until needed.

DNA Analysis of decellularized leaf scaffolds: Samples were first prepared by taking 12.7 mm diameter circular biopsy punches from each lyophilized decellularized leaf. DNA content was quantified to verify complete decellularization. Samples were then cut into 1 mm×1 mm fragments and added to an Eppendorf tube. Samples were flash-frozen in liquid nitrogen and immediately pulverized to reduce the size of the leaf fragments. The DNA content of the samples was measured using a Cyquant DNA assay kit (Thermo Fisher, Waltham, Mass.). Decellularized leaf samples were compared to the DNA standard and non-decellularized leaf samples. Concentrations were measured using a Perkin Elmer Victor3 spectrophotometer.

Primary Satellite Cell Isolation and Culture Conditions

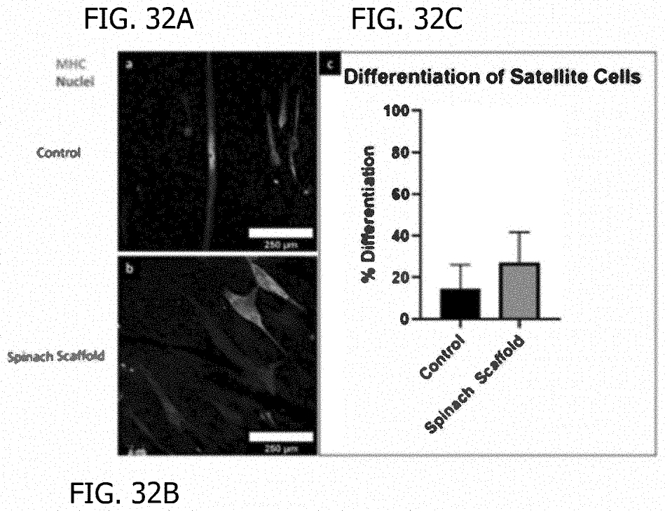

Primary Satellite Cell Isolation: Whole samples of bovine muscle from three different cows (designated cow 1, cow 2, and cow 3) were procured from a local slaughter facility. The muscle samples were kept in separate containers on ice for 30 minutes during transportation from the slaughter facility to the laboratory. Satellite cell isolation began immediately upon arrival (FIG. 30). The entire isolation process was completed inside a laminar flow hood. All instruments and dishes were sterilized in an autoclave (Tuttnauer EZ9-PLUS Steam Sterilizer) prior to isolation. The muscle tissue was placed onto a sterile dish and soaked in digestion medium (DMEM/F12 (Ham's) (Thermo Fisher, Waltham, Mass.), 1% Penicillin/Streptomycin (P/S) (Thermo Fisher, Waltham, Mass.)) for 10 minutes.

Exposure of inner tissue was first done by making a shallow horizontal cut through the center of the muscle. The muscle tissue of either side of this cut was filleted away with a new set of sterile tools to complete interior tissue exposure. Samples were taken from the interior exposed muscle and dissected into approximately 1 mm³ pieces. The samples were then placed in a new sterile dish containing digestion medium (DMEM/F12 (Ham's), 1% (P/S), 10% collagenase (Worthington, Lakewood, N.J.)) and incubated at 37° C. for 1 hour, periodically swirling the dish every 15 minutes. The contents of the dish were transferred to a 50 ml conical tube and allowed to settle to the bottom. The supernatant was removed and passed through a 100 μm sterile cell strainer (VWR, Radnor, Pa.) into a new 50 ml conical tube and spun down at 0.3 rcf for 5 minutes. The tissue pellet was resuspended in 25 ml of sterile rinse medium (DMEM/F12 (Ham's), 1% P/S). Filtration was completed using three 70 μm and three 40 μm cell strainers, spinning down and resuspending the pellet after each filtration. After the final filtration, the pellet was resuspended in 12 ml of growth medium (DMEM/F12 (Ham's), 10% heat-inactivated Fetal Bovine Serum (FBS), 1% P/S, 4 ng/ml FGF2 (ThermoFisher), 2.5 ng/ml HGF (ThermoFisher), 10 ng/ml EGF (ThermoFisher), and 5 ng/ml IGF (ThermoFisher). The isolated cells were incubated overnight at 37° C. and 5% CO₂ to allow cell attachment.

Due to the inherent heterogeneity of the isolated population, it was necessary to enrich the population of satellite cells. Previous works have demonstrated that the satellite cell population can be enriched through differential adhesion pre-plating. This was done by plating the cell suspension on non-tissue culture polystyrene Petri dishes and incubating at 37° C. and 5% CO₂ for 30 minutes to remove unwanted cells from the population prior to subculturing.

Seeding Primary Satellite Cells

Decellularized Spinach Scaffold Preparation: A 12 mm diameter circular punch was used to create scaffolds of uniform size. Scaffolds were then rehydrated using 10 mM Tris Buffer for 15 minutes at room temperature. Scaffolds were sterilized by incubating them in 70% EtOH for 30 minutes in a sterile dish inside of a laminar flow cabinet. After sterilization, scaffolds were rinsed 3 times with sterile PBS, waiting 5 minutes between rinses. Cell seeding was facilitated in a polydimethylsiloxane (PDMS) (DOW Chemical, Midland, Mich.) coated 12-well plate to enhance seeding efficiency. Sterile forceps were used to move each leaf scaffold to a well of the PDMS coated plate. 8 mm diameter cloning wells (VWR, Radnor, Pa.) were placed over the scaffolds to direct the adhesion of seeded cells to a particular area of the scaffold. The cloning wells remained in place for the duration of the seeding process.

Seeding Cells on Decellularized Scaffolds: Approximately 200K cells were deposited directly onto the surface of the scaffold within the cloning well. After a 24-hour cell seeding period, cells that had not adhered were removed by gently rinsing the surface of the leaf with sterile PBS. The growth media inside of the cloning well was replaced, and an additional 1 mL of cell growth media was placed outside of the cloning well to entirely submerge the decellularized leaf.

Viability Assessment of Seeded Satellite cells

Procedure: The satellite cells used in this Example were sourced from 3 different cows. These cells were seeded onto the surface of decellularized leaf scaffolds and compared to a control group of satellite cells grown on gelatin coated glass. Viability was assessed at 2 time points: 7 and 14 days. At the end of each time point, the specimens were fixed in 5% paraformaldehyde and stained using a Live/Dead staining kit (Thermo Fisher, Waltham, Mass.). Cells incubated in 70% EtOH for 30 minutes used as a dead control samples were also stained with Hoechst 33342 (Thermo Fisher, Waltham, Mass.) as a counterstain.

Imaging and Analysis: Samples were imaged using a Leica SP5 point scanning confocal microscope at 20×. Images were taken from random locations across the surface of the leaf. The viability percentage was calculated using the FIJI image processing program to count dead cells and live cells present in each image. A cell was considered dead if the dead marker coincided with the nucleus of the cell. Cells lacking the dead marker were considered viable. The average of these images was used to represent the overall viability of that sample.

Assessment of Differentiation Potential

Procedure: The satellite cells used in this Example were sourced from 3 different cows. These cells were seeded onto the surface of decellularized leaf scaffolds and compared to a control group of satellite cells grown on gelatin coated glass. Once seeded, the satellite cells were submitted to the differentiation protocol as follows. The cells were maintained in growth media (DMEM/F12 (Ham's), 10% heat-inactivated FBS, 1% P/S, 4 ng/ml FGF2, 2.5 ng/ml HGF, 10 ng/ml EGF, and 5 ng/ml IGF) for 2 days. The specimens were then changed to differentiation media containing only 2% heat-inactivated FBS (DMEM/F12 (Ham's), 2% heat-inactivated FBS, 1% P/S, 4 ng/ml FGF2, 2.5 ng/ml HGF, 10 ng/ml EGF, and 5 ng/ml IGF). Differentiation was assessed at two time points: 5 and 12 days after exposure to the differentiation media. At the end of each time point, the specimens were fixed in 5% paraformaldehyde and stained for myosin heavy-chain using MF20 primary antibody (Developmental Studies Hibridoma Bank, Iowa City, Iowa) and Hoechst 33342.

Imaging and Analysis: Samples were imaged using a Leica SP5 point scanning confocal microscope at 20×. Images were taken from random locations across the surface of the leaf. Differentiation percentage was calculated by using FIJI to count nuclei present in each image. A cell was determined to be differentiated if the nucleus coincided with the positive signal of the MHC antibody. All other nuclei were determined to be non-differentiated cells. The average of these images was used to represent the overall differentiation percentage for that sample.

Assessment of Cell Alignment

Procedure: The satellite cells used in this Example were sourced from 3 different cows. These cells were seeded onto the surface of decellularized leaf scaffolds and compared to a control group of satellite cells grown on gelatin coated glass. The satellite cells were maintained in growth media for 2 days. The specimens were then changed to differentiation media. Alignment was assessed at two time points: 5 and 12 days after exposure to the differentiation media. At the end of each time point, the specimens were fixed in 5% paraformaldehyde and stained for f-actin Phalloidin 488 (Life Technologies, Carlsbad, Calif.) and Hoechst 33342.

Imaging and Analysis: Samples were imaged using a Leica SP5 point scanning confocal microscope at 40×. Images were taken from random locations across the surface of the leaf. The alignment was assessed by analyzing the orientation of the cell nuclei and the cytoskeleton. The orientation of the nuclei was measured using the FIJI image processing program by fitting ellipses to each nucleus and measuring the angle of the longest diameter. The OrientationJ algorithm for FIJI was used to measure the orientation of each microfilament within focus in the image. OrientationJ was also used to generate a color survey of each image to help visualize the orientation of each microfilament. The angle distribution of both the nuclei and the cytoskeleton were each generated from this data. Relative alignment can be quantified by comparing the kurtosis of each distribution to another. Because angular data was being analyzed, it was necessary to use angular statistics to analyze the distributions.

The angular data from these images were imported into MATLAB and analyzed using the circstat toolbox. The functions within the circstat tool box were used to calculate the mean vector length, angular standard deviation, and the Kappa value of the distribution. The Kappa value represents the concentration of angle values in the distribution. Kappa values range from 0-1. A value of 0 indicates a perfectly flat distribution, whereas a value of 1 indicates a perfectly aligned distribution. This analysis was done on the nuclei and cytoskeleton independently. The average of these images was used to represent the overall alignment percentage of that sample.

Statistical Analysis

All statistical analysis was done using GraphPad. All the data is expressed as mean±standard deviation. All comparisons were made with either an ordinary one-way ANNOVA or Welch's t-test. A p-value of less than 0.05 was used as the threshold of statistical significance.

### Results

DNA Analysis of Decellularized Leaf Scaffolds

Cyquant analysis of the decellularized samples showed that the decellularization process removed most of the DNA from the leaf material compared to non-decellularized leaf material of the same mass. The decellularized samples had an average DNA content of 72.63±8.03 ng/mg, whereas the non-decellularized leaf samples had an average DNA content of 723.65±80 ng/mg.

Viability Assessment of Seeded Satellite Cells

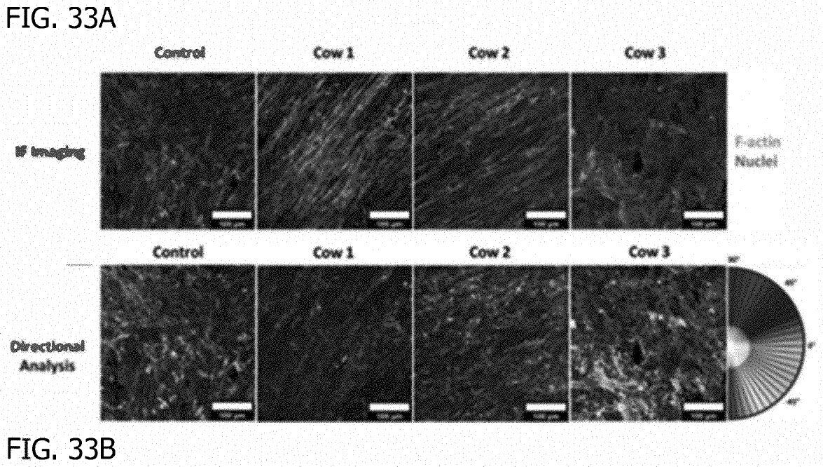

After 7 days of incubation in growth media, the control group cultured on gelatin showed an average of 100% viability. This was also the case for all groups cultured on decellularized leaf scaffolds. After 14 days of incubation in growth media, the control group (FIG. 31A) maintained an average of 100% viability. Samples cultured on decellularized leaf scaffolds also showed strong evidence of overall cell viability. The cells from cows 1 (FIG. 31B), 2, and 3 seeded on decellularized spinach scaffolds had an average viability of 99.87±0.13%, 98.76±0.38%, and 98.95±0.69%, respectively. When compared to the control, all samples grown on the decellularized spinach scaffold showed comparable cell viability (FIG. 31C). A comparison using Welch's t-test indicated that there was no statistically significant difference in viability between cells grown on gelatin or the decellularized leaf scaffolds. A one-way ANNOVA test indicated that there was no significant difference in viability among cells from cow 1, cow 2, and cow 3.

Assessment of Differentiation Potential

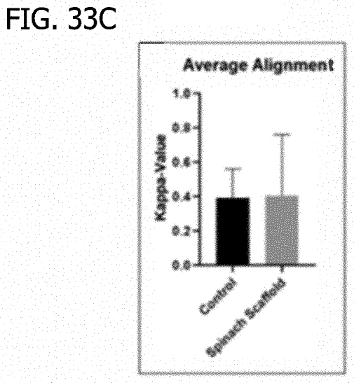

After 7 days of the differentiation protocol, the control group of cells grown on gelatin had a total average differentiation percentage of 7.86±0.92%. Samples from cows 1, 2, and 3 grown on decellularized spinach had an average differentiation percentage of 3.3±1.24%, 0.48±0.48%, and 0% respectively. After 14 days of the differentiation protocol, control group grown on gelatin (FIG. 32A) had a total average differentiation percentage of 19.7±8.12%. Samples from cows 1 (FIG. 32B), 2, and 3 grown on decellularized spinach had an average differentiation percentage of 34.46±11.36%, 29.27±8.34%, and 17.42±2.64% respectively. The samples grown on decellularized spinach were compared to the control group (FIG. 32C) and a Welch's t-test was used to compare cells grown on gelatin and the decellularized leaf scaffolds. The t-test indicated that there was no difference between the cells grown on gelatin or on decellularized scaffolds, with a p-value of 0.193 for 7 days and a p-value of 0.198 for 14 days. A one-way ANNOVA test indicated that there was no significant difference in differentiation percentage among cells from cows 1, 2, and 3 at both time points. Conducting a t-test between 7 and 14 days however, generated a p-value of 0.0002, indicating a strong correlation between timepoints and differentiation percentage.

Assessment of Cell Alignment

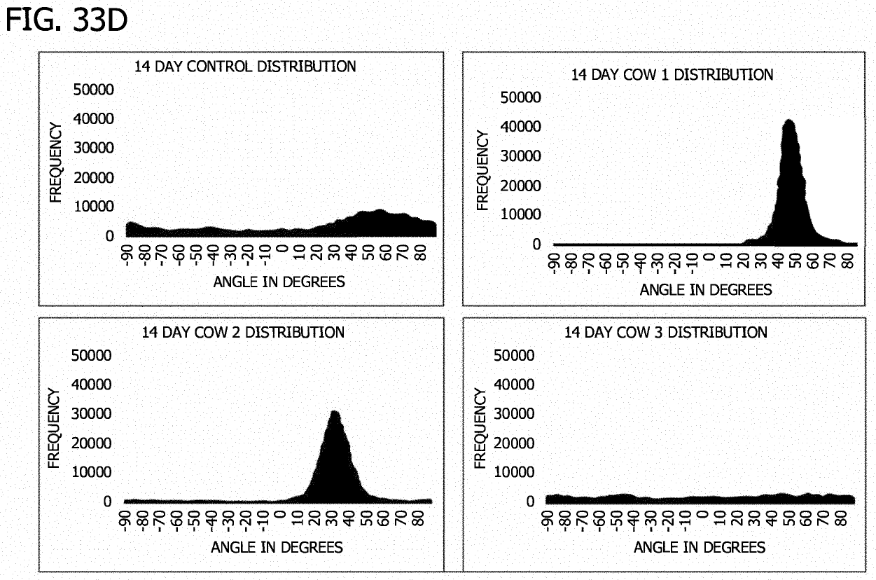

The unprocessed f-actin Phalloidin 488 and hoechst images are illustrated in (FIG. 33A). Color surveys were used to qualitatively assess alignment within the images of each sample (FIG. 33B). Cells grown on gelatin for 7 days of the differentiation protocol showed signs of local alignment within the images, but no indications of overall alignment. Cells grown on the decellularized leaf scaffolds from cow 1 showed relative alignment across images from all technical replicates. However, this result was not shared with the other two biological replicates. Color surveys of cells grown on decellularized leaf scaffolds from cows 2 and 3 showed no signs of alignment. Like samples grown for 7 days, color surveys of cells grown on gelatin for 14 days of the differentiation protocol showed signs of local alignment within regions of the images, but no definitive alignment across the entire image. Samples from cow 1 grown for 14 days of the differentiation protocol on decellularized leaf scaffolds showed strong signs of alignment across entire images in all technical replicates. Cow 2 showed similar alignment in some images, but alignment was inconsistent between technical replicates. Cow 3, on the other hand, continued to show little signs of alignment.

Nuclear alignment and cytoskeleton microfilament alignment were used to quantitatively assess alignment within images of each sample. For all samples, nuclear angle distribution was almost identical to cytoskeleton microfilament angle distributions (FIG. 33D). Samples differentiated for 7 days on gelatin possessed relatively flat distributions with distinct peaks. This was confirmed for cytoskeleton and nuclear alignment with an average kappa value of 0.45±0.063 and 0.09±0.07, respectively. Cells from cow 1 that were cultured on the decellularized leaf scaffold showed marginally better cytoskeleton alignment and significantly better nuclear alignment. Cytoskeletal and nuclear alignment were measured to be 0.64±0.053 and 0.37±0.095, respectively. Cells from cows 2 and 3 grown on decellularized leaf scaffolds for 7 days had kappa values lower than the control group. The average cytoskeleton and nuclear kappa values were 0.134±0.19 and 0.152±0.0501 for cow 2 and 0.167±0.285 and 0.011±0.163 for cow 3.

After 14 days of the differentiation protocol, the cells grown on gelatin showed little change in alignment. The average kappa values for cytoskeletal and nuclear alignment of the control group were measured to be 0.39±0.096 and 0.21±0.13, respectively. Similarly, all samples grown on the decellularized leaf scaffolds for 14 days of the differentiation protocol showed little difference from their 7-day counterparts. Cytoskeletal and nuclear alignment for cow 1 grown for 14 days were measured to be 0.71±0.092 and 0.357±0.0063, respectively. The average cytoskeleton and nuclear kappa values were 0.475±0.177 and 0.202±0.0345 for cow 2 and 0.026±0.079 and 0.051±0.043 for cow 3.

The samples grown on decellularized spinach were compared to the control group (FIG. 33C) and a Welch's t-test was used to compare cells grown on gelatin and the decellularized leaf scaffolds. With p-values of 0.137 and 0.297 for 7 and 14 days respectively, there was no statistically significant difference in relative alignment between cells growth on gelatin and the decellularized leaf scaffolds. A one-way ANNOVA test indicated that there was no statistical significance in relative alignment among cells from cows 1 and 2 at both time points. However, a p-value of 0.0126 from comparison of cows 1 and 3 indicated that there was a significant difference in relative alignment between the groups.

In this Example, the potential of decellularized spinach is demonstrated as a suitable scaffold for development of meat in-vitro. The appeal of using decellularized spinach for meat developments lies not only in its natural vascular network, but also in its edibility and commonality. The edible material eliminates the need to separate the scaffold from the tissue as they will both be consumed. Lastly, spinach is cheap and accessible.

It has been further demonstrated that, after being seeded onto the scaffolds, the primary satellite cells remained viable on the surface of the scaffolds for 14 days with negligible cytotoxic effect. Muscle tissue was also should to be formed on the decellularized spinach from the primary satellite cells.

The results of this Example have shown that the primary satellite cells can differentiate on the surface of decellularized spinach. Analysis of cellular alignment suggests that primary satellite cells do not spontaneously align on the surface of the scaffold. However, on many samples there were instances of high alignment across entire images. It is now believed that it is possible that the local surface topography of the decellularized leaf surface may have had some influence on how the cells arranged themselves in that area. Regions of the leaf that coincide with large vasculature channels of the leaf tend to have crevasses directly above them. Cells that are seeded onto the leaf in these regions will settle into these crevasses. It is possible that local alignment is encouraged along the axis of the channel.

What is claimed is:

1. A method of forming edible animal tissue, comprising:
decellularizing edible plant material to form a scaffold;
seeding the scaffold with animal cells; and
culturing the animal cells to form a grown animal tissue from the seeded scaffold.

2. The method as set forth in claim 1, wherein the plant material is selected from the group consisting of leaf tissue, stem tissue, root tissue, seed, fruit, flower, and combinations thereof.

3. The method as set forth in claim 1, wherein the plant material is from a plant selected from the group consisting of angiosperms, gymnosperms, bryophytes, and algae, and combinations thereof.

4. The method as set forth in claim 1, wherein decellularizing comprises detergent perfusion using at least one of a detergent and enzyme.

5. The method as set forth in claim 1, wherein decellularizing comprises use of one or more detergent selected from the group consisting of sodium hypochlorite (bleach), sodium dodecyl sulfate, sodium hydroxide, ethylenediaminetetraacetic acid (EDTA), Triton X-100, and the like, and combinations thereof.

6. The method as set forth in claim 1, wherein decellularizing comprises use of one or more enzyme selected from the group consisting of lipases, thermolysin, galactosidases, nucleases, trypsin and combinations thereof.

7. The method as set forth in claim 1, wherein seeding comprises a method selected from the group consisting of spraying, coating, submersing cell media, perfusing, and combinations thereof.

8. The method as set forth in claim 1, wherein the animal cells are selected from the group consisting of fibroblasts, myoblasts, myosatellite cells, and combinations thereof.

9. The method as set forth in claim 1, wherein the scaffold further comprises a biomolecule.

10. The method as set forth in claim 1, wherein the biomolecule is selected from the group consisting of nucleic acids, proteins, peptides, growth factors, proteoglycans, and combinations thereof.

11. (canceled)

12. The method as set forth in claim 1, wherein the edible animal tissue is a dried meat material.

13. A system for decellularizing a plant material, the system comprising:
a device for mechanical stirring or shaking, the device capable of holding a container having an open upper end; and
a tiered grate inside the container, located at the lower end, the tiered grate comprising at least two tiers, the first tier having a diameter that is smaller than the second tier.

14. The system as set forth in claim 13, wherein the tiered grate comprises a depth ranging from 0.25 inches to about 4 inches.

15. The system as set forth in claim 13, wherein the device comprises a stir bar, stir bar protector plate, and a stir plate.

16. A method of decellularizing a plant material using the system of claim 11, the method comprising:
placing the plant material into the container;
contacting the plant material with one or more detergent selected from the group consisting of sodium hypochlorite (bleach), sodium dodecyl sulfate, sodium hydroxide, ethylenediaminetetraacetic acid (EDTA), Triton X-100, and the like, and combinations thereof;
mechanically stirring or shaking the plant material in the container; and
allowing plant material to soak in the one or more detergent for a period of from about 30 minutes to about 72 hours.

17. The method as set forth in claim 16, wherein the plant material is selected from leaf tissue, stem tissue, root tissue, seed, fruit, flower, and combinations thereof.

18. The method as set forth in claim 16, wherein the plant material is from a plant selected from the group consisting of angiosperms, gymnosperms, bryophytes, and algae, and combinations thereof.

19. The method as set forth in claim 16 further comprising replacing the detergent with a second detergent, the second detergent being different than the first detergent.

20. The method as set forth in claim 16 further comprising contacting the plant material with one or more enzyme selected from the group consisting of lipases, thermolysin, galactosidases, nucleases, trypsin and combinations thereof.

21. The method set forth in claim 16, wherein the scaffold are further treated with a mineralized coating, where suitable mineral-forming materials may be, for example, calcium, phosphate, carbonate, fluoride, and combinations thereof.
